(19) United States
(12) Patent Application Publication
(10) Pub. No.: US 2017/0065242 A1
(43) Pub. Date: Mar. 9, 2017

(54) Method and System for Analysis of Myocardial Wall Dynamics

(71) Applicant: Circle Cardiovascular Imaging Inc., Calgary (CA)

(72) Inventors: Mihaela Chirvasa, Munich (DE); Xuexin Gao, Calgary (CA); Oskar Skrinjar, Atlanta, GA (US); Kai Philipp Barckow, Berlin (DE); Kelly Cherniwchan, Calgary (CA)

(73) Assignee: Circle Cardiovascular Imaging Inc., Calgary, AB (CA)

(21) Appl. No.: 15/309,344

(22) PCT Filed: May 6, 2015
(86) PCT No.: PCT/CA2015/050399

§ 371 Date: Nov. 7, 2016

Related U.S. Application Data
(60) Provisional application No. 61/989,214, filed on May 6, 2014.

(51) Int. Cl. A61B 6/00; A61B 5/055; G06T 7/00; A61B 6/03

(52) U.S. Cl. CPC A61B 6/5217; A61B 6/032; A61B 5/055; A61B 6/503; G06T 7/0016; A61B 6/466; G06T 2207/30048; G06T 2200/04; A61B 2034/105

(57) ABSTRACT

A method to determine myocardial wall dynamics and tissue characteristics using a method provided to determine myocardial wall dynamics and tissue characteristics using a 3D model of the myocardium. The method comprises generating an epicardial surface and an endocardial surface from a plurality of SAX and LAX slices; identifying a plurality of nodes on the epicardial and endocardial surfaces or in between these surfaces within the myocardium in a reference frame; defining a set of coefficients, each coefficient being associated with the respective location of the corresponding node in a phase; determining the coefficients and in this way determining the model; determining the myocardial wall dynamics in terms of strain values and displacements.

### CROSS REFERENCE TO RELATED APPLICATIONS

This application claims the benefit of priority of U.S. Provisional Patent Application No. 61/989,214 filed May 6, 2014, which is hereby incorporated by reference in its entirety.

### FIELD OF THE INVENTION

The present disclosure relates generally to image processing for understanding, diagnosing, as well as improving existing and developing new treatments for diseases. More particularly, the present disclosure relates to the qualitative and quantitative analysis of myocardial wall dynamics from medical image datasets, such as Computed Tomography (CT) and Magnetic Resonance Imaging (MRI) datasets, derived over a cardiac cycle.

### BACKGROUND OF THE INVENTION

Medical imaging is used as a diagnostic tool as well as an experimental tool to study the anatomy and physiology of humans and other animals. It is also used to guide targeted treatment of some illnesses, for example cancer. Various medical imaging techniques include X-ray, ultrasound, positron emission tomography (PET), magnetic resonance imaging (MRI), and computed tomography (CT).

Digital images obtained through these medical imaging techniques are processed to obtain anatomical and physiological information. One such process is known as strain analysis where medical images are analyzed over a time period to calculate the amount of deformation of a tissue or an organ in a given direction, for example, cardiac strain analysis.

Several techniques have been developed to perform cardiac strain analysis. For example, qualitative and quantitative cardiac strain analysis is done using echocardiography (for example, Tissue Doppler Imaging (TDI) and Speckle Tracking) and MRI (for example, 2D tagged-MRI, Displacement encoding with stimulated echoes (DENSE), Strain Encoding (SENC), and 2D anatomical cine MRI image sets (Feature Tracking)).

There are a number of difficulties associated with strain calculation using the above methods. While echocardiography is known for superior temporal resolution (˜10 ms), it suffers from poor image quality and limited access to all cardiac structures as compared to MRI. However, due to the importance of temporal resolution in analyzing myocardial wall dynamics, echocardiography has been widely used for strain analyses of the myocardium.

RF tagged cine MRI images have also been used due to superior image quality and the ability to visualize a dynamic grid for the myocardial tissue movement (tagging). However, this technique suffers as the RF tags fade over a period of time. Consequently, tracking and quantification of the tags over the entire cardiac cycle becomes difficult resulting in poor temporal resolution. In addition, image acquisition and quantitative post-processing consumes a significant amount of time. Therefore, this technique is not used in routine clinical diagnostics, but is mostly used for research purposes.

Recently, techniques using 2D anatomical cine MRI have been developed that adopt the echocardiography technique of speckle tracking. Instead of tracking the movement of speckles, these methods use regional features of the myocardium, and hence are known as Feature Tracking (for example, TomTec™). The 2D cine MRI technique involve segmenting a region of the myocardium using endocardial and epicardial boundaries and deriving the strain values over the cardiac cycle. However, lack of features in the myocardium as well as through-plane motion of the myocardium makes the method less accurate. Because any point in the myocardium moves through 3-dimensions in a normal cardiac cycle and different parts of the myocardium are visible in the image plane during the cardiac cycle, it is difficult to get a high level of accuracy in static short axis (SAX) or long axis (LAX) slices.

Thus, there remains a need for an improved method to calculate and visualize true myocardial wall dynamics and associated values.

The above information is presented as background information only to assist with an understanding of the present disclosure. No determination has been made, and no assertion is made, as to whether any of the above might be applicable as prior art with regard to the present invention.

### SUMMARY OF THE INVENTION

In an aspect of the present disclosure there is a method provided of determining characteristics of a myocardium using a model of the myocardium and a cine data set, the method comprising: defining a 2D model of the myocardium; determining the 2D model by fitting the 2D model to the cine data set; defining a 3D model of the myocardium; determining the 3D model based on data from the determined 2D model; and performing post processing on the 3D model to determine myocardium characteristics.

In various embodiments, the myocardium characteristics can include tissue characteristics, myocardial dynamics, or myocardial strain.

In an embodiment, determining the myocardium characteristics comprises identifying tissue characteristics. The tissue characteristics can include, for example but is not limited to fibrosis or edema. The tissue characteristics can be an acute or chronic state (e.g. acute or chronic fibrosis).

In some embodiments, the method further comprises rendering a display of a 3D model of the myocardium, the 3D model including a display of strain information on the 3D model.

In some embodiments, the display of strain information comprises a graphical display of magnitudes of strain at various locations on the 3D model.

In various embodiments, the data from the determined 2D model comprises tracked endocardial and epicardial boundaries or the data from the determined 2D model comprises in-slice 2D displacements.

In some embodiments, determining the 2D model comprises: identifying epicardial and endocardial contours in a reference frame of a cine data set; identifying sample points in the reference frame; tracking the sample points through each frame of the cine data set; and determining the 2D model based on the tracked nodes.

In some embodiments, identifying sample points comprises: identifying epi-points, endo-points, and midpoints based on the identified contours of the reference frame; and wherein tracking the sample points through each frame comprises: for each frame in the cine data set: identifying points in the frame corresponding to epi-points and endo-points of a previous frame; transferring midpoints to the frame from the previous frame; and spatially translating the transferred midpoints to improve a match between the identified points in the frame with the corresponding epi-points and endo-points of the previous frame.

In some embodiments, determining the 3D model comprises: defining surfaces to represent the myocardial wall reference frame; setting up node coefficients of a surface model by selecting a set of control nodes from the defined surfaces; selecting a set of myocardium points in a reference frame of the cine data set to serve as 3D sample points; obtaining, for each of the 2D sample points, a set of 2D displacements from the determined 2D model; defining a distance function to measure a total distance between the set of 2D displacements and a set of 2D projection of 3D displacements given by the 3D model; defining a cost function based on the distance function and a smoothness of a displacement field of the 3D model; and determining coefficients of the 3D model by minimizing the cost function.

In some embodiments, determining the 3D model comprises: defining surfaces to represent the myocardial wall at the reference frame; setting up node coefficients of a surface model by selecting a set of control nodes from the defined surfaces; defining standard surfaces using endocardial and epicardial contours from the cine data set; for each frame in the cine data set, generating an estimate of tracked nodes by projecting onto the frame nodes of a previous frame and using the projections as the estimate of the tracked nodes; defining a cost function to measure a distance between the tracked nodes and radial projections of the tracked nodes on the standard surfaces; and determining coefficients of the 3D model by minimizing the cost function.

In another aspect the present disclosure provides a method of determining characteristics of a myocardium using a 2D model of the myocardium and a cine data set. The method comprises: identifying epicardial and endocardial contours in a reference frame of the cine data set; identifying sample points in the reference frame; tracking the sample points through each frame of the cine data set; and performing post processing on the 3D model to determine myocardium characteristics.

In various embodiments, the myocardium characteristics comprise myocardial strain.

In some embodiments, the method further comprises rendering a display of a 2D model of the myocardium, the 2D model including a display of strain information on the 2D model.

In some embodiments, the display of strain information comprises a graphical display of magnitudes of strain on the 3D model.

In various embodiments, identifying sample points comprises: identifying epi-points, endo-points, and midpoints based on the identified contours of the reference frame. In addition, tracking the sample points through each frame can include: for each frame in the cine data set: identifying points in the frame corresponding to epi-points and endo-points of a previous frame; transferring midpoints to the frame from the previous frame; and spatially translating the transferred midpoints to improve a match between the identified points in the frame with the corresponding epi-points and endo-points of the previous frame.

In another aspect, the present disclosure provides a computer readable medium containing statements and instructions for executing any of the above-mentioned methods.

In another aspect, the present disclosure provides a system for determining characteristics of a myocardium using a model of the myocardium and a cine data set. The system includes: a display, an input device; and a processor. The processor is configured and adapted to: define a 2D model of the myocardium; determine the 2D model by fitting the 2D model to the cine data set; define a 3D model of the myocardium; determine the 3D model based on data from the determined 2D model; and perform post processing on the 3D model to determine myocardium characteristics.

In various embodiments, the myocardium characteristics can include tissue characteristics, myocardial dynamics, or myocardial strain.

In an embodiment, determining the myocardium characteristics comprises identifying tissue characteristics. The tissue characteristics can include, for example but is not limited to fibrosis or edema. The tissue characteristics can be an acute or chronic state (e.g. acute or chronic fibrosis).

In some embodiments, the method further comprises rendering a display of a 3D model of the myocardium, the 3D model including a display of strain information on the 3D model.

In some embodiments, the display of strain information comprises a graphical display of magnitudes of strain at various locations on the 3D model.

In various embodiments, the data from the determined 2D model comprises tracked endocardial and epicardial boundaries or the data from the determined 2D model comprises in-slice 2D displacements.

In some embodiments, determining the 2D model comprises: identifying epicardial and endocardial contours in a reference frame of a cine data set; identifying sample points in the reference frame; tracking the sample points through each frame of the cine data set; and determining the 2D model based on the tracked nodes.

In some embodiments, identifying sample points comprises: identifying epi-points, endo-points, and midpoints based on the identified contours of the reference frame; and wherein tracking the sample points through each frame comprises: for each frame in the cine data set: identifying points in the frame corresponding to epi-points and endo-points of a previous frame; transferring midpoints to the frame from the previous frame; and spatially translating the transferred midpoints to improve a match between the identified points in the frame with the corresponding epi-points and endo-points of the previous frame.

In some embodiments, determining the 3D model comprises: defining surfaces to represent the myocardial wall reference frame; setting up node coefficients of a surface model by selecting a set of control nodes from the defined surfaces; selecting a set of myocardium points in a reference frame of the cine data set to serve as 3D sample points; obtaining, for each of the 2D sample points, a set of 2D displacements from the determined 2D model; defining a distance function to measure a total distance between the set of 2D displacements and a set of 2D projection of 3D displacements given by the 3D model; defining a cost function based on the distance function and a smoothness of a displacement field of the 3D model; and determining coefficients of the 3D model by minimizing the cost function.

In some embodiments, determining the 3D model comprises: defining surfaces to represent the myocardial wall at the reference frame; setting up node coefficients of a surface model by selecting a set of control nodes from the defined surfaces; defining standard surfaces using endocardial and epicardial contours from the cine data set; for each frame in the cine data set, generating an estimate of tracked nodes by projecting onto the frame nodes of a previous frame and using the projections as the estimate of the tracked nodes; defining a cost function to measure a distance between the tracked nodes and radial projections of the tracked nodes on the standard surfaces; and determining coefficients of the 3D model by minimizing the cost function.

In another aspect the present disclosure provides a system for determining characteristics of a myocardium using a 2D model of the myocardium and a cine data set. The system includes: a display, an input device; and a processor. The process is configured and adapted to: identify epicardial and endocardial contours in a reference frame of the cine data set; identify sample points in the reference frame; track the sample points through each frame of the cine data set; and perform post processing on the 3D model to determine myocardium characteristics.

In various embodiments, the myocardium characteristics comprise myocardial strain.

In some embodiments, the method further comprises rendering a display of a 2D model of the myocardium, the 2D model including a display of strain information on the 2D model.

In some embodiments, the display of strain information comprises a graphical display of magnitudes of strain on the 3D model.

In various embodiments, identifying sample points comprises: identifying epi-points, endo-points, and midpoints based on the identified contours of the reference frame. In addition, tracking the sample points through each frame can include: for each frame in the cine data set: identifying points in the frame corresponding to epi-points and endo-points of a previous frame; transferring midpoints to the frame from the previous frame; and spatially translating the transferred midpoints to improve a match between the identified points in the frame with the corresponding epi-points and endo-points of the previous frame.

In an aspect of the present disclosure there is a method provided to determine myocardial wall dynamics and tissue characteristics using a 3D model of the myocardium. The method comprises generating an epicardial surface and an endocardial surface from a plurality of SAX and LAX slices; identifying a plurality of nodes on the epicardial and endocardial surfaces or in between these surfaces within the myocardium in a reference frame; defining a set of coefficients, each coefficient being associated with the respective location of the corresponding node in a phase; determining the coefficients and in this way determining the model; determining the myocardial wall dynamics in terms of strain values and displacements.

In an aspect of the present disclosure there is a system provided to determine myocardial wall dynamics and tissue characteristics using a 3D model of the myocardium.

In an aspect of the present disclosure there is a tangible, non-transitory computer-readable medium provided having recorded thereon steps and instructions that when executed by a processor cause a computer to perform the method for determining myocardial wall dynamics and tissue characteristics using a 3D model of the myocardium.

Although the term 3D model is used because of the 3D spatial dimensions, the model can be referred to as a 4D model to account for the dimension of time.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 shows a system according to an embodiment of the present disclosure;

FIG. 2 shows a partial sample of five slices and ten phases of a short-axis (SAX) cine MRI series;

FIG. 3 shows examples of 4-chamber (4Ch), 3-chamber (3Ch), and 2-chamber (2Ch) views for a sample cine MRI series and the corresponding SAX reference slices;

FIG. 4 shows a sample of SAX slices from a cine MRI series and their corresponding location in a LAX reference slice in a 2Ch view;

FIG. 5 is a flowchart of a method for the determination of cardiac parameters based on a model of the myocardium according to an embodiment of the present disclosure;

FIG. 6 is a flowchart of a method for the determination of cardiac parameters based on a 2D model of the myocardium according to an embodiment of the present disclosure;

FIG. 7 shows a SAX slice at end diastole phase with endocardial and epicardial contours as well as mid-nodes located around a mid-curve identified in accordance with an aspect of the present disclosure;

FIG. 8 shows a SAX slice at end systole phase with endocardial and epicardial points as well as mid-nodes located around the mid-curve identified in accordance with an aspect of the present disclosure.

FIG. 9 is an illustration of an image of a chamber of a heart captured using a medical modality;

FIG. 10 is a flow chart diagram illustrating a method for the determination of cardiac parameters based on a 3D model of the myocardium according an embodiment of the present disclosure;

FIG. 11 is a flow chart diagram illustrating a method for the determination of cardiac parameters based on a 3D model of the myocardium according an embodiment of the present disclosure;

FIG. 12 shows an example model of the Left Ventricle (LV) illustrating the various 3D strain directions;

FIG. 13 shows a circumferential strain map obtained from a SAX slice, similar to the slice shown in FIG. 7;

FIG. 14 illustrates a graph showing the average circumferential strain over time of the total myocardial slice (global myocardium);

FIG. 15 illustrates a graph showing average circumferential strain over time of the epi and endo boundary areas of the myocardial slice;

FIG. 16 illustrates a average circumferential strain over time of region of interest (or segment) of the slice as well as corresponding LAX and SAX frames showing the location of the region of interest; and

FIG. 17 illustrates a screen capture of display in accordance with an embodiment of the present disclosure.

### DETAILED DESCRIPTION

Generally, the present disclosure describes methods and systems for image processing for understanding, diagnosing, as well as improving existing and developing new treatments for diseases. More particularly, the present disclosure describes methods and systems for the qualitative and quantitative analysis of myocardial wall dynamics medical image datasets (e.g. 2D cine data sets), such as CT and MRI datasets, derived over a cardiac cycle.

The methods and systems of the present disclosure may be used in many diagnostic and therapeutic areas. For example, the methods and systems of the present disclosure may aid early detection of myocardial insufficiency in a sub-clinical state (or a pre-clinical state when a patient has not been diagnosed with a particular disease or disorder) as well as in both acute and chronic ischemia. Often patients are not treated because all of the functional parameters appear to be normal, such as normal ejection fraction (that is, a fraction of the volume of blood pumped out of the left ventricle (LV) during a cardiac cycle). However, in reality, the patient may be in the initial stages of a disease. Therefore, proper assessment of myocardial wall dynamics may potentially aid in clinically identifying patients with disease or development of disease, when other clinically accepted methods would indicate a normal state.

Another potential clinical/diagnostic use is the detection and quantification of Diastolic Dysfunction where the filling rate of the LV after contraction is impaired. The patient may have a normal ejection fraction as well as end-diastolic (ED) and end-systolic (ES) volumes, but the patient is in a disease state.

In the cases of therapeutics or more specifically interventional or electrophysiology procedure planning, both myocardial wall dynamics and scar tissue registration are important for the eventual success of the treatment.

Consider, for example, an electrophysiology procedure called Cardio-Resynchronization Therapy (CRT). CRT involves implanting a cardiac resynchronization device that resynchronizes the contractions of the heart's ventricles by sending tiny electrical impulses to the heart muscle to assist the heart pump blood throughout the body in a more efficient manner. CRT defibrillators (CRT-D) also incorporate the additional function of an implantable cardiovascular-defibrillator, to quickly terminate an abnormally fast, life-threatening heart rhythm. CRT and CRT-D have become increasingly important therapeutic options for patients with moderate and severe heart failure. Typically, the procedure involves the placement of three leads in the heart, one in the right atrium (RA), one in the right ventricle (RV) (usually at the apex), and one on the epicardial surface of the LV. However, CRT implantations are not extremely successful and only show benefit to the patient in approximately 66% of the cases.

One of the potential causes for failure of CRT has been attributed to scar tissues in the myocardium. Scar tissue (depending on its heterogeneity) changes the electrodynamics of an impulse by either blocking the signal or severely slowing down the propagation of the signal through the tissue. Consequently, a proper synchronization of heart mechanics is not achieved. During a CRT procedure, there is a high probability the leads may be placed in areas of scar tissue leading to potential issues with the proper functioning of the device. Although several electro-physiologists (EP) believe that the electro-anatomical mapping used during current CRT procedures clearly show the areas of scar tissue, the mapping is a very lengthy procedure and does not always display the areas of interest correctly.

The EPs have traditionally paid close attention to electrical mapping as compared to mechanical mapping when identifying the location for the placement of the lead in the LV during a device implantation procedure. The use of mechanical delay information in placing the LV lead in CRT device implantation has been often overlooked because of the requirement for extra imaging studies when it is uncertain the EP can even reach that area with a lead. Also, the EP's are often limited by the coronary vein anatomy. As such, the area of final mechanical delay, which has been shown to be an optimal area for lead placement, is not always reachable.

A study in the United Kingdom called “Targeted Left Ventricular Lead Placement to Guide Cardiac Resynchronisation Therapy: the TARGET study: A Randomised, Controlled Trial,” Khan F Z et al, J Am Coll Cardiol. 2012 Apr. 24; 59(17): 1509-18, provided insight into a number of different areas to address the low rates of efficacy in these device implantation procedures. Mechanical function was analysed using speckle tracking (an echocardiography technique) to measure strain and dyssynchrony. After performing the imaging procedure, an optimized area for lead placement was identified and categorized into three main areas: concordant, adjacent and remote areas.

A Concordant lead placement was in an area that was accessible via the coronary vein anatomy. An Adjacent lead placement was in an area that was one segment away from the optimal area. A Remote lead placement was in an area that was ≧2 segments away from the optimal area. It was shown that the Concordant lead placements had a much greater response rate then the other two.

Thus, there is a need for the identification of the area of final mechanical delay in the myocardium for an effective response using CRT.

Recently, advances have been made in the area of leadless electrodes for electrophysiological devices ranging from pacemakers to CRT-D devices. These leadless electrodes can be placed endocardially and no longer require the coronary venous pathways to implant. Therefore, if the area of final mechanical delay can be identified with a higher probability, leadless electrodes can be implanted with higher accuracy and hence improve the success rates for CRTs. In order to identify the area of interest, myocardial wall dynamics and tissue characteristics are especially valuable markers.

Currently, myocardial wall dynamics analysis methods use echocardiography due to its high temporal resolution. However, as described earlier, due to potential poor image quality, echocardiography-based analysis is not an ideally suited for a proper assessment of myocardial wall dynamics. Cardiac MRI (CMR) Tagged imaging has been gaining popularity due to the higher spatial resolution of CMR to derive segmental strain data. However, CMR Tagged imaging is not practical in a clinical environment. Scanning using these sequences adds unneeded time to the procedure depending on the type of encoding used.

Recent developments in MRI now have provided the ability to obtain datasets with new sequences or phases (number of images per cardiac cycle, for example, 90 phases or images). These developments have led to higher temporal resolution at levels similar to those obtained with echocardiography, yet maintaining high image quality.

The present disclosure describes a method and system for calculating and visualizing myocardial wall dynamics by using 2D anatomical image sets, for example, anatomical cine image sets in SAX and LAX orientation. In addition, the present disclosure describes a method and system for registration and interpolation of the deformation of tissue specific parameters from other MRI or CT image datasets.

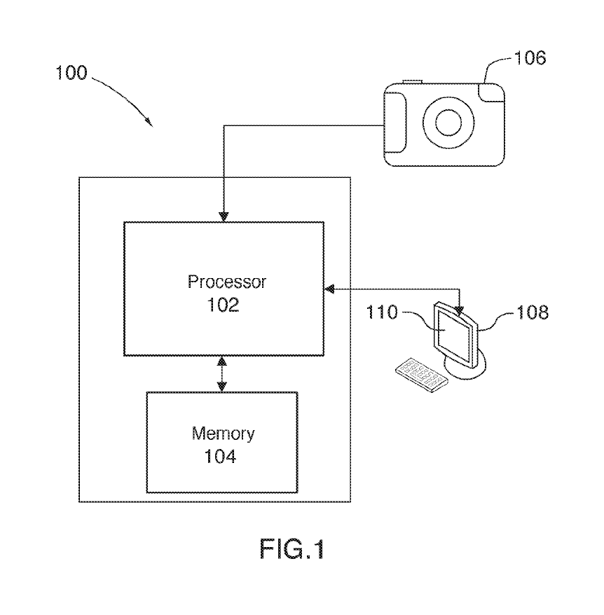

FIG. 1 shows a system 100 according to an aspect of the present disclosure. The system comprises a processor 102 and a memory 104 operatively connected to the processor 102. The processor 102 is configured to receive image series from an image acquisition system 106 (for example, an MRI image acquisition system, CT image acquisition system, or some other medical imaging modality). The processor 102 is further configured to execute the methods of the present disclosure and to display the generated results on the display 110 of terminal 108. Additionally, the processor 102 is configured to receive user inputs from the terminal 108 via keyboards or other suitable input devices, such as mouse, trackpad, touchscreen, tablets etc.

The methods described herein can be used to derive myocardial wall dynamics from any chamber in the heart including the left and right atria as well as the right and left ventricle. For ease of illustration, the method is described in the context of anatomical cine series images of the LV. This is in no way limiting on the applicability of the method to other image sets and/or other chambers of the heart.

In an example embodiment, SAX and LAX images of the LV are used to combine the image sets to form a 4D model (3 dimensional in space with time) to visualize and quantify strain values in all directions.

Generally, the method uses any number of anatomical cine series from a CMR or Cardiac CT (CCT) study to register the 2D cine series to form a 3D model. For example, any number of SAX and LAX cine slices can be used to form a 3D deformable model. The registration can happen in any phase of the cardiac cycle within the anatomical cine series. As the cine series are acquired over many different time points, the method uses the 2D data in the registered 3D model to generate or define a 4D model. The myocardial dynamic values are then calculated to correct for through-plane motion of the 2D images and to provide a more accurate representation of the myocardial dynamic motion.

The 4D multi-parametric model may then be used to identify areas of mechanical delay, myocardial insufficiency and dyssynchrony, as well as the spatial location of the tissue type of interest (for example, scar tissue, edema, or other tissue). Additional parameters derived from T1, T2, or T2* mapping may also be used for tissue characterization.

Furthermore, the method may be used to utilize tissue characteristics from other MR or CT acquisitions (which are usually only acquired in one phase) and interpolate them throughout the cardiac cycle based on the myocardial wall dynamics of the model. As a result, better approximation of the tissue characteristic deformation at any phase within the acquired series may be obtained.

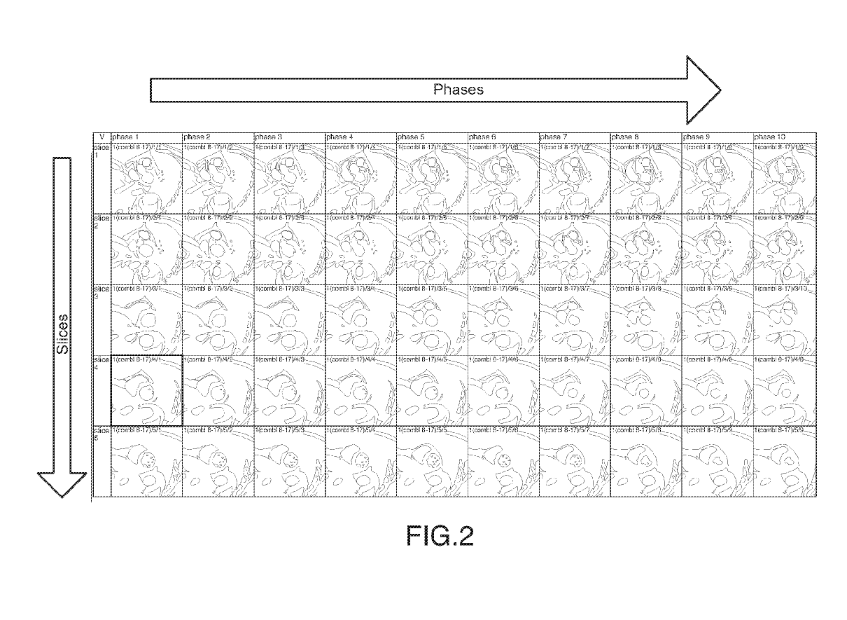

FIG. 2 shows a sample of a cine MRI series including five slices and ten phases. The number of slices and number of phases is generally dependent on the scan protocol (dependent on the instrument acquiring the series). The number of slices is dependent on the slice gap and the number of phases is dependent on the number of time points at which the images are acquired during a cardiac cycle. A cardiac cycle is measured from diastole to systole and back to diastole.

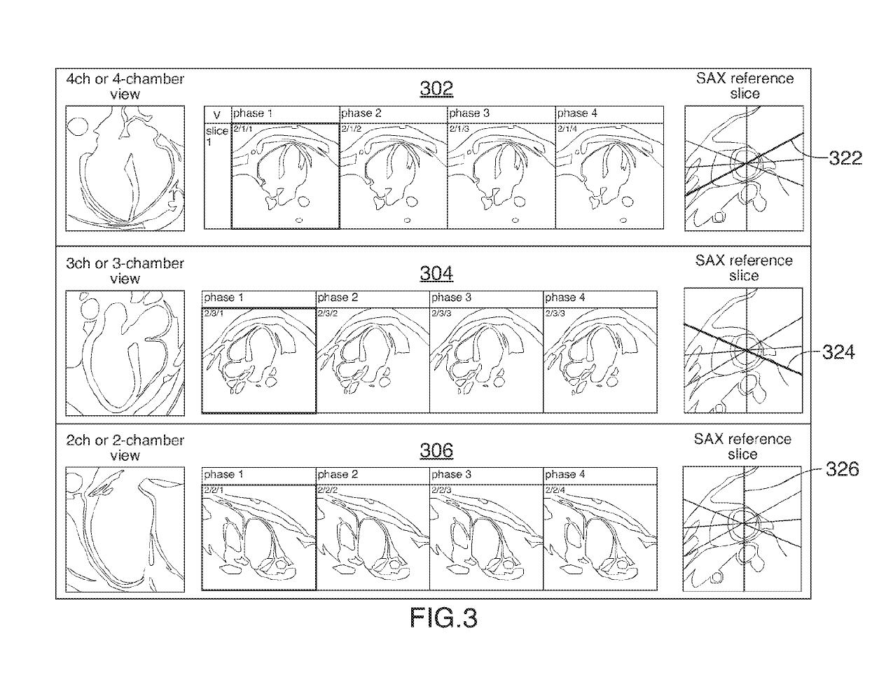

FIG. 3 shows examples of 4-chamber (4Ch) 302, 3-chamber (3Ch) 304, and 2-chamber (2Ch) 306 views for a sample cine MRI series, in this case 4 phases, and the corresponding SAX reference slices (322, 324, and 226). The method of the present disclosure can process any number of slices obtained from any of these views to determine myocardial wall dynamics and tissue characteristics.

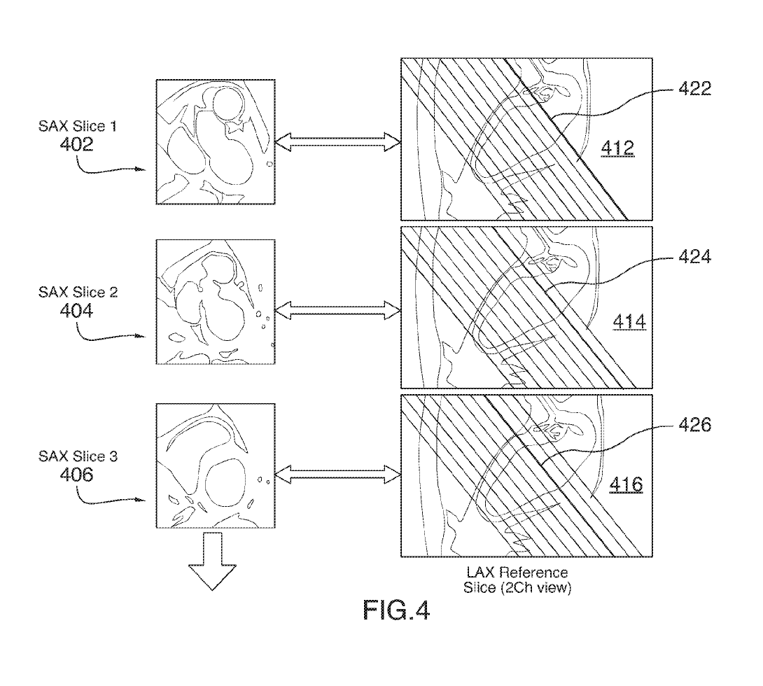

FIG. 4 shows multiple SAX slices and their corresponding locations in a LAX reference slice in a 2Ch view of a sample cine MRI series. Specifically, 3 SAX slices (402, 404, and 406) are shown for purposes of illustration; however, it should be noted that a cine MRI series will generally include a much larger number of slices. Images 412, 414, and 416 are instances of the same LAX reference image (2Ch view). The lines in the LAX reference image illustrates where the SAX images are located in the LAX slice. Accordingly, each instance of the LAX reference image highlights a specific line that corresponds to one of the SAX slices. In particular, line 422 corresponds to SAX slice 402, line 424 corresponds to SAX slice 404, and line 426 corresponds to SAX slice 406.

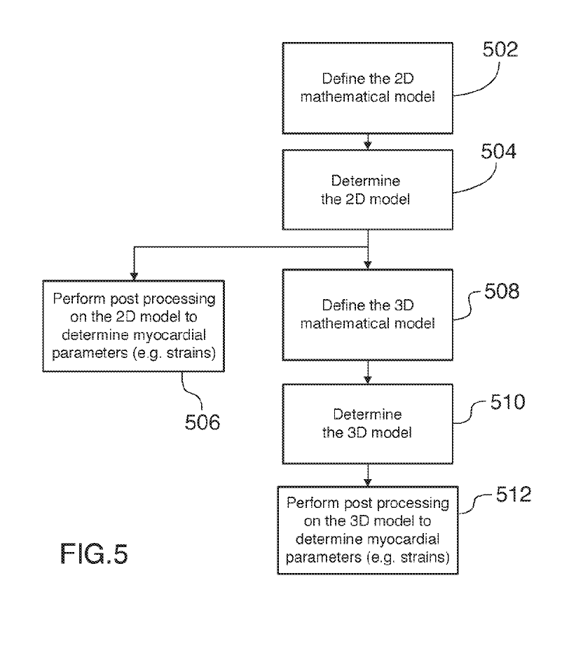

FIG. 5 is a flowchart of a method for the determination of cardiac parameters based on a model of the myocardium according to an aspect of the present disclosure. The method may be carried out by software executed by, for example, a processor, such as processor 102 of system 100 FIG. 1. Coding of software for carrying out such a method is within the scope of a person of ordinary skill in the art given the present description. The method may contain additional or fewer processes than shown and/or described, and may be performed in a different order. Computer-readable code executable by at least one controller or processor, such as for example processor 102 or a different processor, to perform the method may be stored in a computer-readable medium, such as a non-transitory computer-readable medium.

The method can include a 2D model as well as a 3D model of the myocardium in accordance with an aspect of the present disclosure. Although the models are referred to as 2D and 3D based on their spatial dimensions. However, there is also a time dimension and therefore the models may be referred to as having an additional dimension.

The method includes defining a 2D mathematical model of the myocardium (502). The model may be of a portion of the myocardium such as the myocardium corresponding to the LV. The 2D model is then determined (504). For example, in an embodiment, the model is determined by fitting the model to 2D cine data sets. Detailed examples of how this performed are discussed in greater detail below.

Once the 2D model has been determined, the method can include performing post processing in order to determine various characteristics of the myocardium that may be of interest (506). Alternatively, the method can include generating and using a 3D model to determine the myocardial characteristics (508, 510, and 512).

The method can include defining a 3D mathematical model of (a portion of) the myocardium (508). For example, the model may be of the LV. In an embodiment the assumption is made that the myocardium deformation is fully determine by the deformation of a set of myocardium points (nodes) chosen in the myocardium wall (e.g. the LV wall when the LV is being modeled). The displacement field from the reference frame to the current frame is modeled as a linear combination of radial basis functions, each weighted by a coefficient. In an embodiment, each coefficient is associated with the position of a node in the current frame (one-to-one correspondence). In the reference frame, all the coefficients are zero.

The model is then determined by fitting the 3D model to the 2D results (510). Determining the model means determining the position of the nodes in each frame.

Post processing is then performed in order to determine various characteristics of the myocardium that may be of interest (512). This can include determining myocardial parameters such as cardiac strains and displacements, as well as other information.

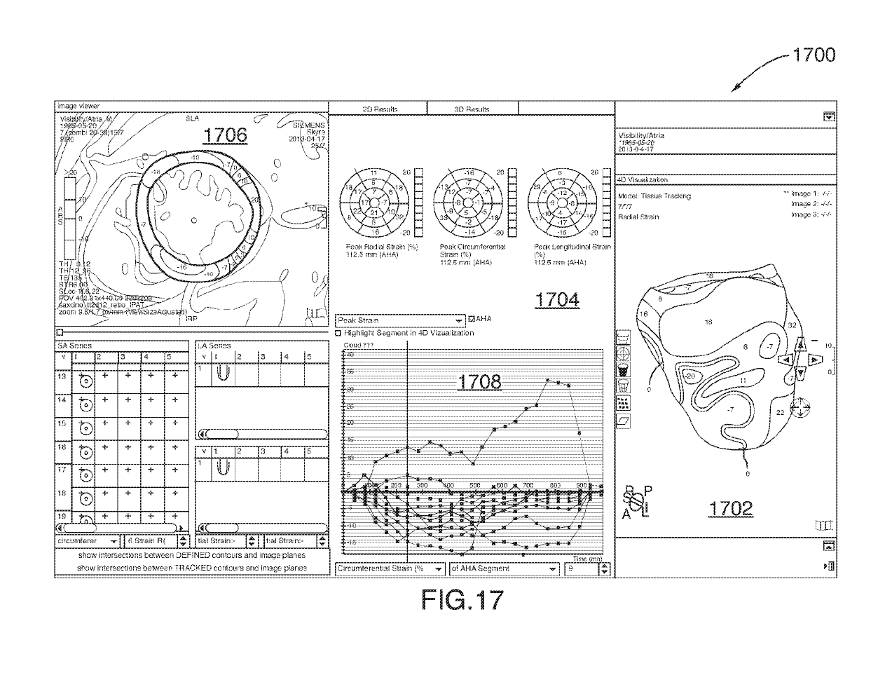

The method can also include displaying information based on the model and the determined parameters (e.g. strain parameters). In particular, the display can include visualizations (in both 2 dimensions and 3 dimensions) of the model with displayed strain characteristics as well as graphs or charts. This information may prove useful to a medical profession (e.g. a cardiologist or other clinician) or researchers. In particular, by displaying the 3D model and incorporated information (e.g. strain information) allows a person to see the physical location of the strain on the myocardium, which could prove useful in diagnosing disease or structural irregularities in the myocardium, for research purposes generally, or other purposes. An example of such a display is provided in FIG. 17 as well as other figures of this disclosure.

The 3D model (and the 2D model as well) accurately models the tissue characteristics of the myocardium and therefore allows one to predict the behavior of the tissue over time (e.g. over a cardiac cycle). This modeled behavior (and tissue characteristics) can be compared to “normal” or “expected” behavior and tissue characteristic (e.g. the behavior and characteristics of healthy heart) and can thereby be used to identify abnormalities or pathology in the myocardium. This can include, but is not limited to, identification of areas of fibrosis or edema or other conditions, whether they are acute or chronic. Accordingly, in some embodiments, the methods include comparing the data obtained from either the model (either the 2D model or the 3D model) to “normal” or “expected” data and identifying areas of concern (e.g. tissue characteristics such as fibrosis) based on differences between the two.

In an example embodiment, the set of coefficients associated with the nodes is computed for each phase or frame in the series (for the entire cardiac cycle). Once the nodes are determined (from their coefficients), the dynamics of the myocardium can be determined, that is a 3D model in time (or a 4D model) of the myocardium is obtained. From the 4D model, locations of initial points at any time, strains, torsions and other parameters of the myocardium can be determined.

The strain (%), strain rate (%/t), displacement (mm), velocity (mm/t), torsion (deg/cm), torsion rate (deg/cm/t) as well as the minimum, maximum, and average of these values can be determined from the above method. In addition, for each of the circumferential, radial and longitudinal directions, the peak strain, time to peak strain, peak systolic strain rate, peak diastolic strain rate, peak displacement and peak velocity can also be determined.

In an example embodiment, a set of connected points may be used instead of individual endo and epi points. The set of connected points may be tracked over the various image frames throughout the cardiac cycle.

In an example embodiment, the tracking of the connected points (or any points on the endo/epi surface) may be used to visualize the deformation of the heart during the cardiac cycle. In an example embodiment, the points may be connected using tessellation of the unit sphere using triangles (other shapes may also be used) and projecting the vertices of these triangles on the endo/epi surfaces of the reference frame. In addition, tracking the vertices of these triangles tessellated surfaces may also be obtained for each phase. Tessellation may also be used to generate or define endo/epi contours on all the images by intersecting the tracked, tessellated endo/epi surfaces with image planes. The endo/epi contours generated or defined in the 3D model may then be validated by a user.

In an example embodiment, the myocardial and myocardial chamber volumes in ED or ES may be determined using the epicardial and endocardial volumes, that is, as a difference between the two volumes. In addition, the associated ejection fraction (EF), mass, and stroke volumes (SV) may also be automatically determined.

The method of the present disclosure also allows for user interaction. If the 2D tracking failed in one phase (for example, due to through-place motion), the user can manually identify contours, which can then be used for the registration step. For example, nodes for the phase where 2D tracking failed can be adjusted such that the mapped points best match the manual contours. The 3D results for this phase can then be accordingly updated.

Based on the calculated strain and displacement values, and derivatives thereof, relative areas of interest can now be automatically calculated and mapped. As in the previous example of CRT, the above values allow computation and visualization of the area of final mechanical delay. Combined with other MRI or CT series (for example, acquired for visualization and quantification of tissue characteristics), a full model of the myocardial area of interest can be generated showing mechanical delay/dysynchrony and key tissue characteristics in spatial locations over the entire cardiac cycle. In cases where the information of the tissue characteristics is available only for one time frame, the myocardial displacement can be used to interpolate the deformation of the volume of interest (for example, scar tissue) for the visualization of the entire cardiac cycle.

Myocardium Model

Some embodiments disclosed herein relate to methods and systems for generating a 3D myocardium model. As mentioned elsewhere in this disclosure, the 3D model can be of a chamber of the heart, such as the LV. In an embodiment, the method includes fitting an incompressible deformable model of the wall of a portion of the heart to individual image slices (including but not limited to, any combination of short axis slices, long axis slices, and arbitrarily oriented slices) over the cardiac cycle and then regenerating a 3D displacement field which is nearly incompressible. The portion of the heart could be, for example, but is not limited to a chamber of the heart such as the left ventricle. An incompressible model is used is because myocardial tissue is nearly incompressible.

In an embodiment, the method is divided in 2 major steps:

1. generating a 2D deformable model as a 2D version of the 3D incompressible deformable model described in Bistoquet, A., Oshinski, J., Skrinjar, O., Left Ventricular Deformation Recovery from Cine MRI Using an Incompressible Model, September 2007, which is incorporated herein in its entirety. Determine the model using image feature tracking.

2. Generating a 3D deformable model and determining it using the 2D tracking results described above.

2D Model

Reference is first made to the 2D model. In an embodiment, the 2D model is generated or defined as a 2D deformable model as a 2D version of the 3D incompressible deformable model described in Bistoquet and this model is fitted on each frame of the image sequence using data obtained from feature tracking.

In an embodiment, the generation of the 2D model is based on the assumption that the deformation of the model is determined by the deformation of the mid-curve of the model. In the case of LV wall slices, the mid-curve is the curve that goes through the middle of the LV wall: in the case of short-axis slices the mid-curve is a closed curve and in the case of long-axis slices the mid-curve is an open curve. The mid-curve is represented by nodes that are interpolated to define the curve in between nodes.

Let m(u)=(x(u),y(u)) represent the mid-curve in the reference frame. The curve is in parametric form with u being the parameter. Let \hat{n}(u) represent a unit vector normal to the mid-curve at point m(u). Let γ represent distance from point m(u) in direction \hat{n}(u). Thus, a point can be defined by a pair of numbers (u,γ), which are called curvilinear coordinates, i.e. its location is

r(u,γ)=m(u)+γ\hat{n}(u) (1)

Let M(u)=(X(u),Y(u)) represent the mid-curve point in the current frame corresponding to the point m(u) in the reference frame (note that the two have the same parameter u). [Note: Lowercase symbols refer to the reference frame while uppercase symbols refer to the current frame.] The point in the current frame corresponding to point r(u,γ) in the reference frame is given by

R(u,γ)=M(u)+Γ(u,γ)\hat{N}(u) (2)

where \hat{N}(u) is a unit vector normal to the mid-curve at point M(u) and Γ(u,γ) is the distance of point R(u,γ) to the mid-curve. The distance Γ(u,γ) is determined such that the mapping from the reference to the current frame (which maps point r(u,γ) to point R(u,γ)) is incompressible. In the 2D case, this means that the mapping is area preserving, i.e.

da=dA (3)

where da is the infinitesimal area in the reference frame at point r(u,γ) corresponding to infinitesimal changes in u and γ, and dA is the corresponding area in the current frame. Since

$$d a = \vert \frac{\partial r}{\partial u} \times \frac{\partial r}{\partial γ} \vert \cdot d u \cdot d γ and d A = \vert \frac{\partial R}{\partial u} \times \frac{\partial R}{\partial γ} \vert \cdot d u \cdot d γ \qquad ( 4 )$$

the relations (1)-(4) lead to the following equation in Γ:

$$\sqrt{{( \frac{d x}{d u} )}^{2} + {( \frac{d y}{d u} )}^{2} γ} + \frac{1}{2} \frac{\frac{{d}^{2} x}{d {u}^{2}} \frac{d y}{d u} - \frac{{d}^{2} y}{d {u}^{2}} \frac{d x}{d u}}{{( \frac{d x}{d u} )}^{2} + {( \frac{d y}{d u} )}^{2}} {γ}^{2} = \sqrt{{( \frac{d X}{d u} )}^{2} + {( \frac{d Y}{d u} )}^{2}} Γ + \frac{1}{2} \frac{\frac{{d}^{2} X}{d {u}^{2}} \frac{d Y}{d u} - \frac{{d}^{2} Y}{d {u}^{2}} \frac{d X}{d u}}{{( \frac{d X}{d u} )}^{2} + {( \frac{d Y}{d u} )}^{2}} {Γ}^{2} \qquad ( 5 )$$

To summarize, the transformation is defined by the mid-curve m(u) in the reference frame and the corresponding mid-curve M(u) in the current configuration. To map a point from the reference frame to the current frame, the first step is to obtain its curvilinear coordinates (u,γ) in the reference configuration (based on eq. (1)), then solve for Γ(u,γ) in eq. (5), and finally obtain the location of the point in the current frame using eq. (2).

In an embodiment, to determine the model means to find the locations of the mid-nodes in each frame.

Deformable Model Fitting

Once the LV wall is segmented in the reference frame, its boundary is known and one can define the mid-curve and distribute nodes over the mid-curve. To fit the model to any other (current) frame, the mid-curve nodes need to be moved in the current frame until the corresponding (according to the model mapping) image information between the reference and current frame match. The LV wall in the anatomical cine cardiac MR image slices general does not have clear and reliable image features that can be used to determine the deformation inside the wall (this is why tagged MRI has been developed); rather it appears as a relatively homogenous region with close-to-constant image intensity. The only reliable image features of the LV wall are its boundaries.

For this reason the method first determines the LV wall boundary in the current frame by feature tracking and then deforms the model (i.e. moves the nodes in the current frame) to fit this boundary.

Feature Tracking Procedure:

To minimize the effect of out of plane motion, the feature tracking is done from the previous frame (instead of reference frame) to the current.

The boundaries from the previous frame are copied onto the current frame.

For each boundary point in the previous frame and in the current frame, a small rectangular window is defined centered at the given boundary point, with the sides in the normal and tangential directions.

The window is slid in the normal direction over the image in the current frame (in the previous frame stays fixed) and as this is done, the image information stored in the windows from the current and previous frames are used to generate a mean squared displacement (msd) profile. If the minimum of the profile is pronounced, then this minimum defines the boundary point in the current frame. If it is not, the point is for the moment discarded.

All the boundary points in the current frame computed from minima of the msd-profiles serve as anchor points for determining the other undetermined boundary points.

That is, a boundary point which could not be determined from the msd profile is determined by interpolation on an ellipse using two neighbour anchor points.

After the boundaries in the current frame have been determined by FT, the next step is to find the nodes for which the mapped boundaries from the reference frame match the boundaries determined by FT as close as possible while still generating a smooth transformation. This is achieved by finding the mid-curve node positions in the current frame that minimize:

$$COST = \sqrt{\frac{1}{N} {\sum }_{i = 1}^{N} {\vert {R}_{i}^{M} - {R}_{i}^{FT} \vert}^{2}} + λ \sqrt{\frac{1}{N} {\sum }_{i = 1}^{N} [ {( \frac{d {E}_{r}}{d u} ( {R}_{i}^{M} ) )}^{2} + {( \frac{d {E}_{c}}{d u} ( {R}_{i}^{M} ) )}^{2} ]} \qquad ( 6 )$$

In the above formula N is the number of boundary points, Rᵢ^{M} and Rᵢ^{FT} are the i-boundary points in the current frame computed by mapping and correspondingly, by feature tracking, λ is a weight that controls the relative contribution of the two terms, Eᵣ is the radial strain and E_{c} is the cross-radial strain (both defined in the section “Myocardial Strain Computation”) evaluated at each boundary point. The first term in the above equation measures the mismatch between the mapped and feature tracked boundary and the second term measures the smoothness of the transformation.

The optimization is performed using a variant of the Powell's method, which is disclosed in Press, W., Flannery, B., Teukolsky, S., and Vetterling, W, Numerical Recipes in C: The Art of Scientific Computing, 2nd Ed., 1992, which is incorporated herein in its entirety. Each node is moved in the positive and negative normal (to the mid-curve) direction and in the positive and negative tangential (to the mid-curve) direction and the node position that minimizes COST is kept. The distance the node is moved in the normal/tangential direction is specified by parameter delta. The nodes are moved over and over again until COST can no longer be reduced. Then the normal and tangent deltas are cut in half, and the optimization is repeated until COST can no longer be reduced. Then the normal and tangent deltas are again cut in half, and the optimization is repeated. Different values of the deltas are called scale levels (or levels of refinement). The number of scale levels is controlled by a parameter.

Merging Forward and Backward Deformation Recoveries:

The model is fitted from frame to frame, starting with the reference frame and continuing until the last frame. During this process fitting errors accumulate and consequently the model is more accurately fitted to the frames from the beginning of the image sequence then to the frames from the end of the image sequence. However, since the LV wall motion is periodic, the method also fits the model in the backward direction: starting from the reference (first) frame, the model is fitted to the last frame, then to the second to last frame, and so on until the second frame. Finally, the forward and backward deformation recoveries are combined to obtain a deformation recovery that has a better accuracy than either the forward deformation recovery alone or the backward deformation recovery alone.

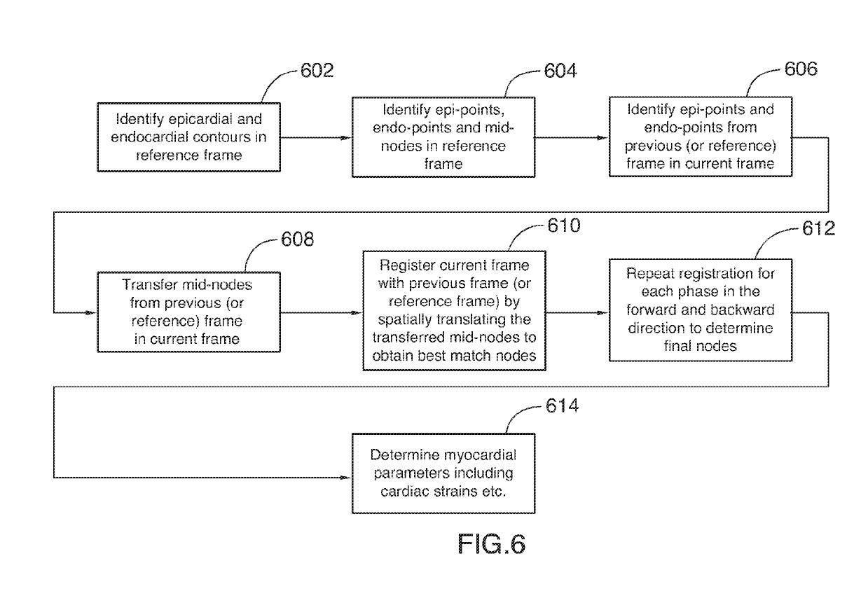

FIG. 6 is a flowchart of a method for the determination of cardiac parameters based on a 2D model of the myocardium according to an aspect of the present disclosure. The method may be carried out by software executed by, for example, a processor, such as processor 102 of system 100 FIG. 1. Coding of software for carrying out such a method is within the scope of a person of ordinary skill in the art given the present description. The method may contain additional or fewer processes than shown and/or described, and may be performed in a different order. Computer-readable code executable by at least one controller or processor, such as for example processor 102 or a different processor, to perform the method may be stored in a computer-readable medium, such as a non-transitory computer-readable medium.

In an aspect of the present disclosure, as in Bistoquet, the ED phase (of frame) of the cardiac cycle is used as a reference frame. Once the epicardial and endocardial contours are identified (602), a set of points (to be tracked) is chosen on these contours and the midcurve is defined (604). Registration of the frames is done iteratively from phase to phase (or frame to frame) throughout the cardiac cycle. The registration of the current frame with the previous frame (or the reference frame) may be done in two steps: a feature tracking step and a mapping step.

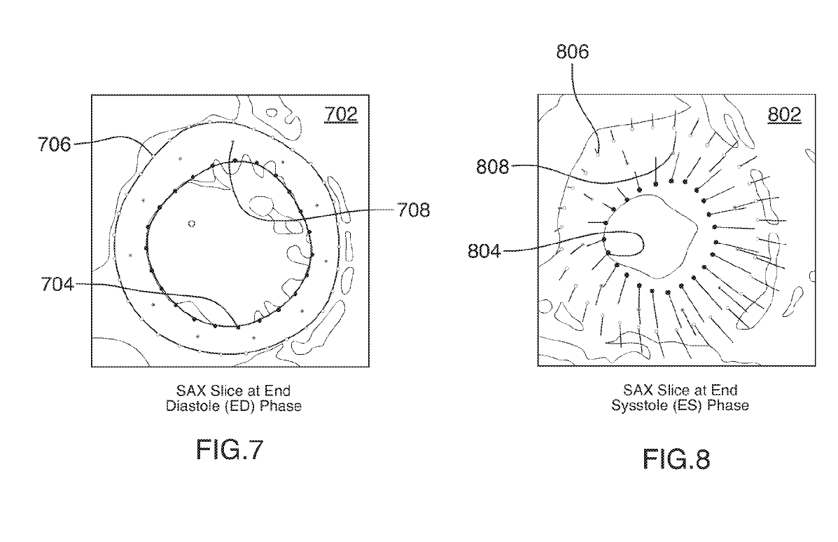

In the feature tracking step, points or features on the epi and endo curves (epi and endo points) from the previous frame are identified in the current frame (feature tracked points) (606). In FIG. 7, the epi and endo points are shown as dots along the epicardial and endocardial contours. In FIG. 8, spatial displacements of these points are shown using lines connected to the epi and endo points (appearing as streaks in FIG. 8).

In the mapping step, the mid-nodes from the previous frame (or the reference frame) are transferred to the current frame and are spatially translated (608). For each spatial translation, the spatial configuration of the nodes defines a mapping of the endo and epi points from the reference frame to the current frame (mapped points).

The nodes configuration for which the best match between the feature-tracked points and the mapped points is obtained, defines the nodes in the current frame. The epi and endo points from the previous frame (or the reference frame) are thus mapped to the current frame using the best match nodes to complete the registration of the current frame (610).

A constraint used in the registration step (that is a combination of feature tracking and mapping steps) is that the myocardium is nearly incompressible and that local area is preserved, that is, the area of myocardium for the slice (for example, the area between the epicardial and endocardial contour) is preserved throughout the cardiac cycle. In other words, the circumferential area shown in FIG. 8 remains substantially constant in all phases of the cardiac cycle.

The feature tracking and mapping steps (that is, the frame registration process) are repeated for each phase or frame in the series (for the entire cardiac cycle) to obtain the midnodes. In an example embodiment, the iterative process of registration is done in forward and backward directions and the final nodes are obtained by combining the nodes-result of these two processes (612). Once the final nodes are determined, various dynamic quantities can be computed (614), for example, cardiac strains etc. For the SAX circumferential direction, the strain (%), strain rate (%/t), displacement (mm), velocity (mm/t), torsion (deg/cm), torsion rate (deg/cm/t) as well as the minimum, maximum, and average of these values can be determined. For the SAX and LAX radial direction as well as the LAX longitudinal direction, the strain (%), strain rate (%/t), displacement (mm), velocity (mm/t), including the minimum, maximum, and average of these values can be determined. In addition, for each of the circumferential, radial and longitudinal directions, the peak strain, time to peak strain, peak systolic strain rate, peak diastolic strain rate, peak displacement and peak velocity can also be determined.

Because the true anatomical function of the heart is in a 3D space, the calculation of strain values in 2D has the potential to yield incorrect results. If a 2D slice is acquired throughout a cardiac cycle, different parts of the myocardium move into and out of plane due to true motion in a 3D space. This movement in and out of plane is known as through-plane motion and may not be captured in 2D imaging, whether in tagged MRI or anatomical cine series as examples.

FIG. 7 shows a SAX slice 702 at end diastole phase (ED) with an endocardial contour 704 and an epicardial contour 706, as well as mid-nodes 708 identified in accordance with an aspect of the present disclosure. FIG. 8 shows a SAX slice 802 at end systole (ES) phase with endocardial points 804 and epicardial points 806 as well as mid-nodes 808 identified in accordance with an aspect of the present disclosure.

A circumferential map is obtained from the SAX slice once the endocardial and epicardial contours have been identified. FIG. 13 (discussed below) illustrates a SAX slice with a circumferential map. A mid-curve (not shown in FIG. 7 and FIG. 8) is identified in substantially the central region of the circumferential map and mid-nodes (nodes or points on the mid-curve) are derived from the mid-curve. The mid-nodes (708 and 808) are identified in between the epicardial and endocardial contours in FIGS. 7 and 8. Any number of mid-nodes can be identified and processed based on processing capacity and efficiency of the image processing system. Segmentation of the myocardium in the ED phase, identification of the epicardial and endocaridal contour as well as the mid-curve are described in detail in “Left Ventricular Deformation Recovery From Cine MRI Using an Incompressible Model,” A. Bistoquet et al, IEEE Transactions of Medical Imaging, Vol. 26, No. 9, September 2007, 1136-1153 (“Bistoquet”), which is incorporated herein by reference in its entirety. Bistoquet further describe a method to identify mid-nodes on the mid-curve and to define a mid-surface of the LV wall.

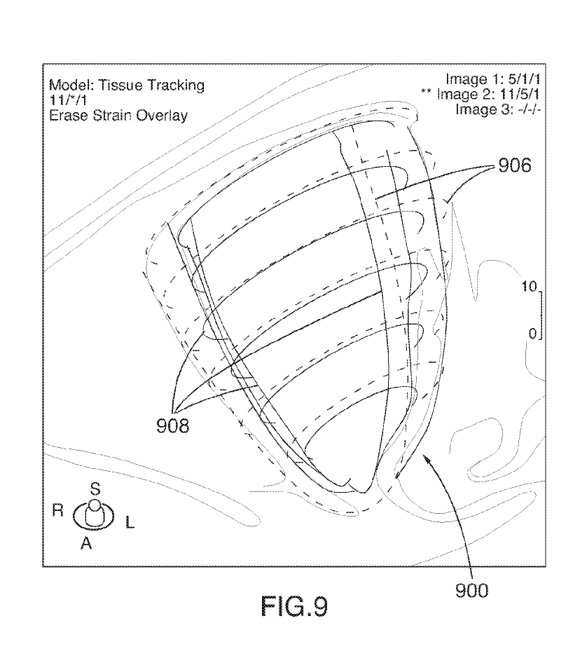

FIG. 9 is an illustration of an image of a chamber of a heart (specifically, the left vertical is show in this example) captured using a medical modality. FIG. 9 shows multiple epicardial 906 and endocardial 908 contours derived from multiple SAX images and LAX images identified in a 3D space, in accordance with a method of the present disclosure.

In an embodiment of the present disclosure, the SAX and LAX images are registered in order to compensate for the spatial misalignment due to, for example, but not limited to, patient movement (or other factors) during image acquisition. In some embodiments, the registration of the images is done in two steps: a contour matching step and an intensity matching step.

In an embodiment, in the contour matching step, all LAX images are fixed spatially and each SAX image is iteratively translated to minimize the difference between the contour intersections from LAX and SAX images. For each translation, the SAX image is intersected with each LAX image in a line as they are not parallel. The epicardial (or endocardial) points lie on the intersection line of two different images (SAX and LAX) and should have a minimal difference in location when the SAX images are well-aligned. In other embodiments, the SAX images are fixed and the LAX images are translated.

In an embodiment, in the intensity matching step, one LAX image is chosen to be fixed initially and other images are iteratively translated to maximize the correlation between the intensity profiles from intersected images. To begin, each LAX image is intersected with all the other images (LAX and SAX) in lines and the image pixel intensity profiles on the intersection line from two images should be similar when the images are well-aligned. The similarity could be quantified by various mathematical models, for example, but not limited to, spectral coherent, cross-covariance, and cross-correlation. For example, a cross-correlation method may be used to determine the similarity. In an embodiment, the LAX image with highest correlation with other images is selected as the most suitable image to be used as an anchor for image alignment. After that, the other LAX image(s) is/are translated iteratively until a maximal correlation of intensity profiles at intersections is found. Similar to what was mentioned above in relation to the with respect to the contour matching step, in some embodiments, the role of the LAX and SAX images can also be reversed for the intensity matching step.

In an embodiment, a threshold could be set to determine whether the maximal correlation is sufficiently high for the translation to be effective. In such an embodiment, if the threshold is not reached, then the translation is canceled. Once all LAX images have been registered, they are then fixed spatially as anchors so that each SAX image may be also aligned by evaluating intensity profile correlation.

These steps associated with the description of FIG. 9 are done as a precursor to the 3D methods described below.

3D Model

Two different basic embodiments of the method for generating the 3D myocardium model will be described herein. Both of these basic models can utilize the same 3D deformable model. However, these two basic embodiments differ in which 2D tracking results are used for determining the unknown coefficients. Given that different 2D tracking results are used by each of these embodiments, each of the embodiments also differs in the details of the method.

The first of these embodiments will be referred to as the “displacement-based 3D model method”, which uses the in-slice 2D displacements (from the 2D method described above) as inputs to determine the coefficients. The second of these embodiments will be referred to as the “surface-based 3D model method”, which uses the in-slice tracked endocardial and epicardial boundaries (from the 2D method described above) as inputs to determine the coefficients. Additional detail on how these inputs are used will be discussed below in greater detail.

The 3D methods attempt to determine the myocardial wall dynamics by modeling the near-incompressibility of the myocardium. As mentioned above, in some embodiments, both methods can utilize the same 3D model. This 3D model will now be briefly discussed.

A myocardium point at position r in reference frame will be mapped to another frame at position T(r). The difference u(r)=T(r)−r represents the displacement field.

An example of a 3D deformable model of the myocardium that can be used by some embodiments discussed herein, can be defined in the following way:

First, a set of M points on the region that is to be modelled (which may be for example, but is not limited to, the LV wall) is selected in the reference frame. These points are to be identified as nodes. Their positions are arbitrary but known rⱼ for j=1, . . . , M.

Second, the assumption is made that the displacement field can be expanded as a linear combination of M scalar basis functions centered at node positions in reference frame rⱼ each weighted by a coefficient cⱼ which need to be determined. For the scalar basis functions we use a radial basis function

$$f ( r ) = {e}^{\frac{- {\vert r \vert}^{2}}{2 {α}^{2}}}$$

centered at nodes rⱼ, for j=1, . . . , M, where α controls how fast the function decays. Thus, the model transformation is given by the formulas:

T(r)=r+u(r) with u(r)=Σⱼ₌₁^{M}ƒ(r−rⱼ)cⱼ (7)

The scalar radial functions describe the positions of an arbitrary myocardium point relative to the nodes in the reference frame. The coefficients are frame dependent, being associated with the positions of the nodes in that frame. Determining these coefficients for each frame determines the myocardial dynamics within this model.

Note that if the coefficients in one frame are known, then the nodes positions are also known by (7). The opposite is also true. Accordingly, if the nodes positions in a frame are known, then their displacements are known and (7) becomes a linear system of 3M equations and 3M unknowns for the coefficients cⱼ. As mentioned above, both the displacement based method and the surface based method can be used to determine the nodes. Each of these methods will be discussed in turn below in greater detail.

Displacement-Based Method

In the displacement-based method, the coefficients are found by matching the in-slice displacements obtained with the 2D method with the projected 3D displacements field onto the slices. The term matching, as used in the preceding sentence, indicates that the regenerated 3D displacement field, when projected onto the slices, is close (and in some embodiments as close as possible) to the corresponding in-slice displacements. In order to achieve this, the minimization problem for the sum of the squared in-plane distances between the projected displacement field and the point in-slice displacements is solved for each frame. The problem has a closed solution because it reduces to determining the coefficients by solving a linear system of 3M equations and 3M unknowns. The mathematical details are discussed below.

As mentioned above, the regenerated 3D displacement field (7), when projected onto the slices of points pᵢ, should be as close as possible to the corresponding in-slice displacements uᵢ. Let âᵢ and \hat{b}ᵢ represent two unit vectors that together with \hat{n}ᵢ represent an orthonormal basis. Note that vectors âᵢ and \hat{b}ᵢ are in the same slice as point i and they represent an orthonormal basis for the 2D space of the image slice. Thus, the sum of squared in-plane distances between the projected displacement field and the point in-slice displacements is:

$$\begin{matrix}{E}_{match} = \frac{1}{N} {\sum }_{i = 1}^{N} [ {( {\hat{a}}_{i} \cdot {d}_{i} )}^{2} + {( {\hat{b}}_{i} \cdot {d}_{i} )}^{2} ] \\ where & ( 8 ) \\ {d}_{i} = u ( {p}_{i} ) - {u}_{i} & ( 9 )\end{matrix}$$

By defining ƒᵢⱼ=ƒ(pᵢ−rⱼ) and by combining (7), (8), and (9) it follows that

$${E}_{match} = \frac{1}{N} {\sum }_{i = 1}^{N} [ {( {\hat{a}}_{i}^{T} {\sum }_{j = 1}^{M} {f}_{ij} {c}_{j} - {\hat{a}}_{i}^{T} {u}_{i} )}^{2} + ( {\hat{b}}_{i}^{T} {\sum }_{j = 1}^{M} {f}_{ij} {c}_{j} - {\hat{b}}_{i}^{T} {u}_{i} ) ] \qquad ( 10 )$$

By minimizing E_{match}, i.e. by finding vector coefficients cⱼ that minimize E_{match}, one obtains a displacement field whose projection closely matches the in-slice displacements but that is typically not smooth. To ensure the resulting displacement field is smooth we minimize the following:

E=E_{match}+λ₁Eₛₘₒₒₜₕ₁+λ₂Eₛₘₒₒₜₕ₂,

where Eₛₘₒₒₜₕ₁ and Eₛₘₒₒₜₕ₂ are measures of smoothness of the displacement field and λ₁ and λ₂ are parameters controlling the relative importance of the two terms, respectively. The smoothness terms at L uniformly spaced points over the myocardium sₗ are evaluated. For the first smoothness term, the following expressions are used:

$${E}_{smooth 1} = {F}_{x} + {F}_{y} + {F}_{z} \\ with \text{:} {F}_{x} = \frac{1}{L} {\sum }_{l = 1}^{L} {\vert \frac{\partial u}{\partial x} \vert}^{2} ( {s}_{l} ) {F}_{y} = \frac{1}{L} {\sum }_{l = 1}^{L} {\vert \frac{\partial u}{\partial y} \vert}^{2} ( {s}_{l} ) {F}_{z} = \frac{1}{L} {\sum }_{l = 1}^{L} {\vert \frac{\partial u}{\partial z} \vert}^{2} ( {s}_{l} ) \qquad ( 11 )$$

For the second smoothness term, the following expressions are used:

$${E}_{smooth 2} = {S}_{x} + {S}_{y} + {S}_{z} \\ with \text{:} {S}_{x} = \frac{1}{L} {\sum }_{l = 1}^{L} {\vert \frac{{\partial }^{2} u}{\partial {x}^{2}} \vert}^{2} ( {s}_{l} ) {S}_{y} = \frac{1}{L} {\sum }_{l = 1}^{L} {\vert \frac{{\partial }^{2} u}{\partial {y}^{2}} \vert}^{2} ( {s}_{l} ) {S}_{z} = \frac{1}{L} {\sum }_{l = 1}^{L} {\vert \frac{{\partial }^{2} u}{\partial {z}^{2}} \vert}^{2} ( {s}_{l} ) \qquad ( 12 )$$

The goal is to minimize E, which can be achieved by requiring that:

$$\frac{\partial E}{\partial {c}_{m}} = 0 for m = 1 , … , M . \qquad ( 13 )$$

Since

$$\frac{\partial E}{\partial {c}_{m}} = \frac{\partial {E}_{match}}{\partial {c}_{m}} + {λ}_{1} \frac{\partial {E}_{smooth 1}}{\partial {c}_{m}} + {λ}_{2} \frac{\partial {E}_{smooth 2}}{\partial {c}_{m}} ,$$

below we discuss the derivatives of the match and smoothness terms.

From

$$\frac{\partial {E}_{match}}{\partial {c}_{m}} = \frac{2}{N} {\sum }_{i = 1}^{N} {f}_{𝒥} [ {\hat{a}}_{i} {\hat{a}}_{i}^{T} ( {\sum }_{j = 1}^{M} {f}_{ij} {c}_{j} - {u}_{i} ) + {\hat{b}}_{i} {\hat{b}}_{i}^{T} ( {\sum }_{j = 1}^{M} {f}_{ij} {c}_{j} - {u}_{i} ) ] \qquad ( 14 )$$

it follows that

$$\frac{\partial {E}_{match}}{\partial {c}_{m}} = \frac{2}{N} {\sum }_{j = 1}^{M} [ {\sum }_{i = 1}^{N} {f}_{ij} {f}_{𝒥} ( {\hat{a}}_{i} {\hat{a}}_{i}^{T} + {\hat{b}}_{i} {\hat{b}}_{i}^{T} ) ] {c}_{j} - \frac{2}{N} {\sum }_{i = 1}^{N} {f}_{𝒥} ( {\hat{a}}_{i} {\hat{a}}_{i}^{T} + {\hat{b}}_{i} {\hat{b}}_{i}^{T} ) {u}_{i} \qquad ( 15 )$$

It can be shown that the matrix âᵢâᵢ^{T}+\hat{b}ᵢ\hat{b}ᵢ^{T} can be directly computed from \hat{n}ᵢ as âᵢâᵢ^{T}+\hat{b}ᵢ\hat{b}ᵢ^{T}=I−\hat{n}ᵢ\hat{n}ᵢ^{T}, where I is an identity matrix. The matrix P(\hat{n})=I−\hat{n}\hat{n}^{T} is known as the projection matrix since P(\hat{n})v represents the projection of vector v to the plane defined by its unit normal vector \hat{n}. Thus, (15) can be rewritten as

$$\frac{\partial {E}_{match}}{\partial {c}_{m}} = \frac{2}{N} {\sum }_{j = 1}^{M} [ {\sum }_{i = 1}^{N} {f}_{ij} {f}_{𝒥} P ( {\hat{n}}_{i} ) ] {c}_{j} - \frac{2}{N} {\sum }_{i = 1}^{N} {f}_{𝒥} P ( {\hat{n}}_{i} ) {u}_{i} \qquad ( 16 )$$

From (11) it follows that:

$$\begin{matrix}\frac{\partial {E}_{smooth 1 }}{\partial {c}_{m}} = \frac{\partial {F}_{x}}{\partial {c}_{m}} + \frac{\partial {F}_{y}}{\partial {c}_{m}} + \frac{\partial {F}_{z}}{\partial {c}_{m}} \\ with & ( 17 ) \\ {F}_{x} = \frac{1}{L} {\sum }_{l = 1}^{L} [ {\sum }_{j = 1}^{M} \frac{\partial f}{\partial x} ( {s}_{l} - {r}_{j} ) {c}_{j}^{T} ] [ {\sum }_{k = 1}^{M} \frac{\partial f}{\partial x} ( {s}_{l} - {r}_{k} ) {c}_{k} ] & ( 18 )\end{matrix}$$

and similar relations for F_{y} and F_{z}. Let

$${FX}_{lj} = \frac{\partial f}{\partial x} ( {s}_{l} - {r}_{j} ) .$$

### Then,

$${F}_{x} = {\sum }_{j = 1}^{M} {\sum }_{k = 1}^{M} {fx}_{jk} {c}_{j}^{T} {c}_{k} where {fx}_{jk} = \frac{1}{L} {\sum }_{l = 1}^{L} {FX}_{lj} {FX}_{lk} \qquad ( 19 )$$

From (19) it follows that:

$$\frac{\partial {F}_{x}}{\partial {c}_{m}} = 2 {\sum }_{k = 1}^{M} {fx}_{mk} {c}_{k} \qquad ( 20 )$$

From (12) it follows that:

$$\begin{matrix}\frac{\partial {E}_{smooth 2}}{\partial {c}_{m}} = \frac{\partial {S}_{x}}{\partial {c}_{m}} + \frac{\partial {S}_{y}}{\partial {c}_{m}} + \frac{\partial {S}_{z}}{\partial {c}_{m}} \\ with & ( 21 ) \\ {S}_{x} = \frac{1}{L} {\sum }_{l = 1}^{L} [ {\sum }_{j = 1}^{M} \frac{{\partial }^{2} f}{\partial {x}^{2}} ( {s}_{l} - {r}_{j} ) {c}_{j}^{T} ] [ {\sum }_{k = 1}^{M} \frac{{\partial }^{2} f}{\partial {x}^{2}} ( {s}_{l} - {r}_{k} ) {c}_{k} ] & ( 22 )\end{matrix}$$

and similar relations for S_{y} and S_{z}.

Let

$${SX}_{lj} = \frac{{\partial }^{2} f}{\partial {x}^{2}} ( {s}_{l} - {r}_{j} ) .$$

### Then,

$${S}_{x} = {Σ}_{j = 1}^{M} {Σ}_{k = 1}^{M} {sx}_{jk} {c}_{j}^{T} {c}_{k} where {sx}_{jk} = \frac{1}{L} {Σ}_{l = 1}^{L} {SX}_{lj} {SX}_{lk} \qquad ( 23 )$$

From (23) it follows that

$$\frac{\partial {S}_{x}}{\partial {c}_{m}} = 2 {\sum }_{k = 1}^{M} {sx}_{mk} {c}_{k} \qquad ( 24 )$$

Combining (13), (16), (17) and (20), (21) and (24) yields the result:

$$\begin{matrix}{\sum }_{j = 1}^{M} [ {\sum }_{i = 1}^{N} {f}_{ij} {f}_{I} P ( {\hat{n}}_{i} ) + {λ}_{1} NI ( {fx}_{mj} + {fy}_{mj} + {fz}_{mj} ) + {λ}_{2} NI ( {sx}_{mj} + {sy}_{mj} + {sz}_{mj} ) ] cj = {\sum }_{i = 1}^{N} {f}_{I} P ( {\hat{n}}_{i} ) {u}_{i} & ( 25 ) \\ for m = 1 , … , M . & \end{matrix}$$

Equation (25) represents a system of 3M equations and 3M unknowns (vector coefficients cⱼ). Once cⱼ are determined, the displacement field can be evaluated at any point by using (7).

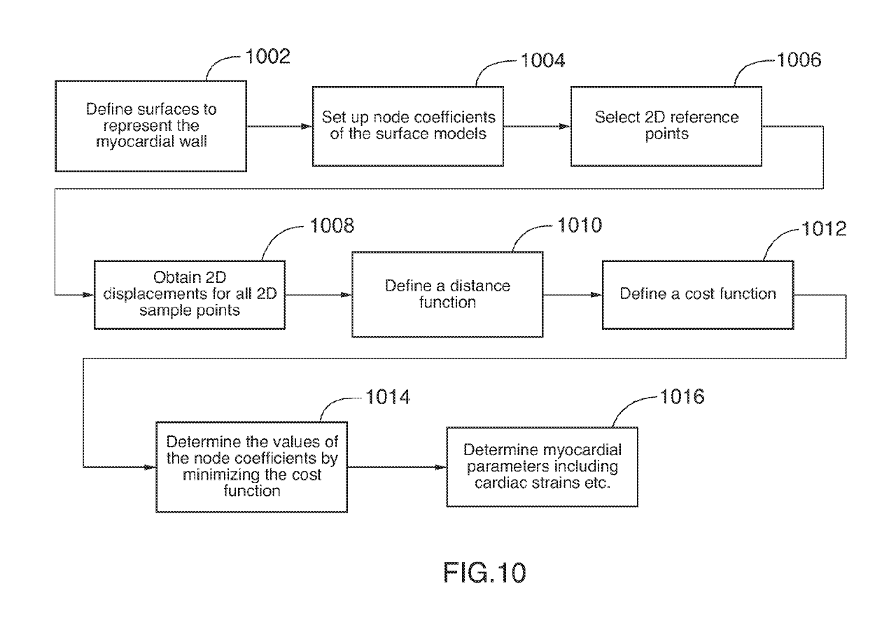

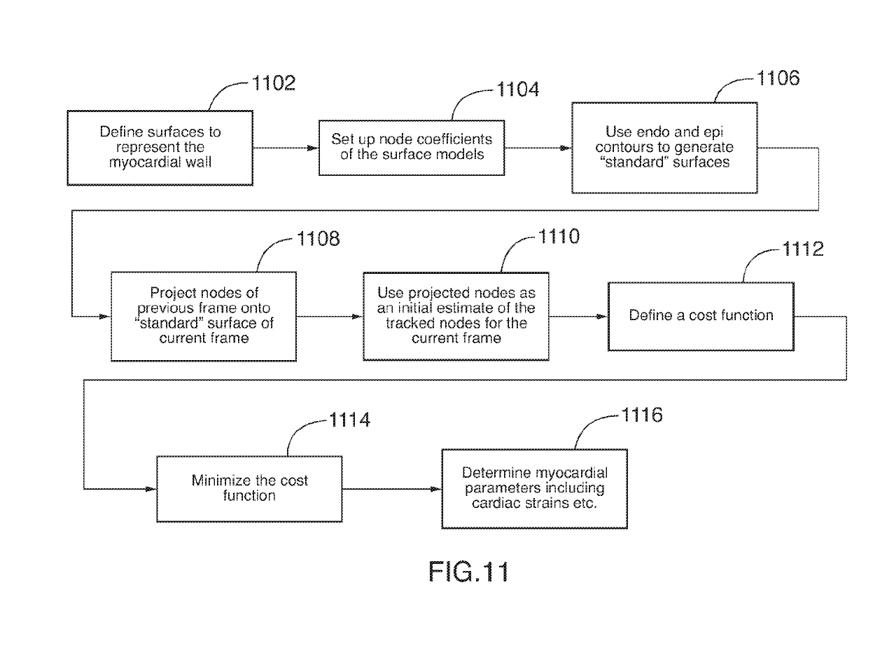

Reference is now made to FIG. 10, which is a flow chart diagram of the displacement-based method, according to an embodiment of the present disclosure. The method may be carried out by software executed by, for example, a processor, such as processor 102 of system 100 FIG. 1. Coding of software for carrying out such a method is within the scope of a person of ordinary skill in the art given the present description. The method may contain additional or fewer processes than shown and/or described, and may be performed in a different order. Computer-readable code executable by at least one controller or processor, such as for example processor 102 or a different processor, to perform the method may be stored in a computer-readable medium, such as a non-transitory computer-readable medium.

The method includes, at the reference fame, generating surfaces to represent the myocardial wall based on user segmented contours (1002). The method further includes selecting a set of control points (nodes) from the surfaces to setup the node coefficients of the surface model (1004).

A set of myocardium points in the reference frame are selected to serve as 2D sample points (for example, all the myocardium points within slice, in the reference frame, centered at the image pixels) (1006). From the computed 2D model, the 2D displacements for all of the 2D sample points are obtained (1008). A distance function is defined to measure the total distance between the in-slice 2D displacements of the samples points and the 2D projections of the 3D displacements given by the 3D model (1010). A cost function is defined which includes the defined distance function as well as smoothness terms for the 3D displacement field (1012). The method further includes determining the values of the node coefficients by solving a liner system that minimizes the defined cost function (1014). Determining the node coefficients allows the model to be determined. Based on the node coefficients (i.e. based on the determined model), the 3D displacement of any point (at any other frame) can be derived based on the determined node coefficients or other myocardial parameters can be determined (1016).

Surface-Based Method

In the surface-based method, for each frame, the tracked endocardial and epicardial contours from all the slices are interpolated using pseudo-thin-plate interpolation to define endocardial and epicardial surfaces (the interpolation method is described in greater detail below under the heading Appendix Smooth Surface Model). Optionally, if the 2D tracking contours in an image are not “satisfactory”, one can use user defined (e.g. user drawn) contours in their place.

The endocardial and epicardial surfaces defined above are considered to be “standard” surfaces. A basic idea of this approach is to determine the coefficients cⱼ for which the mapped endocardial and epicardial surfaces from the reference frame to the current frame using (7) are as close as possible to the “standard” surfaces existing in that frame. The matching mapped-standard surfaces are based on a minimization criterion of the squared sum of the distances between the mapped surface points and their radial projections on the “standard” surfaces. The minimization problem as stated does not have a closed solution (as in the case of the “displacements-based” method discussed above) and we solve it numerically via an iterative process.

The method is iterative both in time and in space. It is iterative in time because in order to determine the coefficients in the current frame, the coefficients in the previous frame are used to generate an initial estimate for the coefficients in the current frame. More precisely, the initial estimate of nodes is set to be the nodes from the previous frame radially projected on the “standard” surfaces of the current frame. The method is iterative in space, because (in an embodiment of the method) keeping the frame fixed (current frame), the method starts from this initial estimate and tunes it (iteratively) until the matching reaches the desired tolerance. The following discussion will focus on this iterative process in space.

For tuning the parameters the method uses point set registration (also known as point matching). In computer vision and pattern recognition, point set registration (or point matching) is the process of finding a spatial transformation that aligns two point sets.

One of the sets of points is regarded as the “moving” model point set while the other is the “static” scene. The term “moving”, in this context, means changing the model point set iteratively due to adjusting the transformation parameters from iteration to iteration. The model point set and the static scene are not required to have the same number of points.

In the case of the method described herein, the “moving” model is represented by the mapped endocardial and epicardial surface's points in the current frame, while the static scene is represented by the points of the “standard” surfaces of the current frame. The 3D mapping formula (7) provides the transformation for alignment of the two sets.

To solve the minimization problem within this point matching approach an embodiment of employs the Levenberg-Marquardt method. This is a method that is used for solving nonlinear least square problems. Its typical use is in the least squares curve fitting problem: Given a set of N empirical datum pairs of independent and dependent variables (xᵢ,yᵢ), optimize the parameters c of the model curve ƒ(x,c) so that the sum of the squares of the residues (cost function) E(c)=Σᵢ₌₁^{N}[yᵢ−ƒ(xᵢ,c)]² is minimized.

In the present case, the problem can be stated as follows: given a set of N^{endo} points on the endocardial reference frame surface Pᵢ^{endo} and a set of N^{epi} points on the epicardial reference frame surface, optimize the parameters c of the model function (7) such that the sum of the squares of the differences between the tracked points, T(Pᵢ^{endo},c) and T(Pᵢ^{epi},c) and their radial projections on the standard surfaces S^{goldEndo}T(Pᵢ^{endo},c) and S^{goldEpi}T(Pᵢ^{epi},c) are minimized:

$$E ( c ) = {\sum }_{i = 1}^{{N}^{endo}} {[ {S}^{goldEndo} T ( {P}_{i}^{endo} , c ) - T ( {P}_{i}^{endo} , c ) ]}^{2} + {\sum }_{i = 1}^{{N}^{epi}} {[ {S}^{goldEpi} T ( {P}_{i}^{epi} , c ) - T ( {P}_{i}^{epi} , c ) ]}^{2} \qquad ( 26 )$$

The number of parameters to be determined is 3M. Since the count of nodes on each reference surface, in an embodiment, is of the order 100 (other embodiments may use other amounts), and there are 2 reference surfaces (one for endo and one for epi), each node has 3 coordinates, there will be altogether around 600 parameters to be determined. This is computationally expensive.

In order to reduce the computation time, in an embodiment, the following measures are implemented:
instead of looking at the coefficients c as parameters to be determined, the method instead considers the node positions in the current frame as unknown quantities. Recall that the node positions and the coefficients can be obtained directly one from another.
during the iteration process, the moving of the nodes is restricted to be within the standard surfaces. That the degree of freedom is reduced for one node from 3 to 2.
the minimization problem is broken into 2: one for endocardial nodes optimization and one for epicardial nodes optimization.

In other words, the following sums are minimized separately:

$$\begin{matrix}E ( {c}^{endo} ) = {\sum }_{i = 1}^{{N}^{endo}} {[ {S}^{goldEndo} T ( {P}_{i}^{endo} , {c}^{endo} ) - T ( {P}_{i}^{endo} , {c}^{endo} ) ]}^{2} & ( 27 ) \\ E ( {c}^{epi} ) = {\sum }_{i = 1}^{{N}^{epi}} {[ {S}^{goldEpi} T ( {P}_{i}^{epi} , {c}^{epi} ) - T ( {P}_{i}^{epi} , {c}^{epi} ) ]}^{2} & \end{matrix}$$

where c^{endo} and c^{epi} are the set of coefficients corresponding to the endocardial and epicardial nodes, respectively, in the current frame.

These are examples only. There are other options for the cost functions. For example, instead of using the difference: mapped point—its projection on the standard surface, one can use the relative distance.

In this way, a tolerance level can be set up which measures (e.g. in percentage) the matching of the mapped and standard surfaces.

Reference is now made to FIG. 11, which is a flow chart diagram of the surface-based method, according to an embodiment of the present disclosure. The method may be carried out by software executed by, for example, a processor, such as processor 102 of system 100 FIG. 1. Coding of software for carrying out such a method is within the scope of a person of ordinary skill in the art given the present description. The method may contain additional or fewer processes than shown and/or described, and may be performed in a different order. Computer-readable code executable by at least one controller or processor, such as for example processor 102 or a different processor, to perform the method may be stored in a computer-readable medium, such as a non-transitory computer-readable medium.

The method includes, at the reference fame, generating surfaces to represent the myocardial wall based on user segmented contours (1102). The method further includes selecting a set of control points (nodes) from the surfaces to setup the node coefficients of the surface model (1104).

The method also includes using the endocardial and epicardial contours to define the “standard” surfaces (1106). The contours can be tracked contours or user defined contours. Then, for each frame (other than the reference frame), the nodes of the previous frame are radially projected onto the “standard” surface of the current frame (1108). The projected nodes are used as an initial estimate of the tracked nodes for the current frame (1110). A cost function is defined to measure the distance between the tracked surface points and their radial projections on the “standard” surface (1112).

The cost function is then minimized or brought to a value within certain defined criteria (1114). In an embodiment, this is done by using the Levemberg-Marquardt method to iteratively move the nodes within the “standard” surface until the cost function is minimized or meets the defined criteria. This provides the coefficients of the model and the model is then determined. This can then be used to determine 3D strain parameters of the myocardium to determine the myocardial wall dynamics (1116).

Alternative to Minimization Problem

Instead of using Levemberg-Marquardt method as described above, some embodiments use, for example, a variant of the Powell's method used in the determining the 2D model, adapted for 3D. That means that each node is moved in the positive and negative radial/circumferential/longitudinal direction (to the surface on which it belongs), and the node position that minimizes the cost function is kept. The distance the node is moved in the radial/circumferential/longitudinal direction is specified by some parameters “delta”. In an embodiment, the nodes are moved over and over again until the cost can no longer be reduced. Then the deltas are cut in half, and the optimization is repeated until again the cost can no longer be reduced. Different values of the deltas are called scale levels (or levels of refinement). The number of scale levels can be controlled by a parameter.

Myocardial Strain Computation

Myocardial strain is computed as a Lagrangian finite strain relative to the reference frame. Let F denote the deformation gradient tensor. Then the Lagrangian tensor is:

E=½(F^{T}F−I) (28)

where I is the identity matrix. The Lagrangian strain in unit direction \hat{v} is

s(\hat{v})=\hat{v}^{T}E\hat{v} (29)

2D Strain

In the 2D case, if the mapping from the reference to the current frame in Cartesian coordinates is given by functions X(x,y) and Y(x,y), then the deformation gradient tensor is

$$F = [ \begin{matrix}\frac{\partial X}{\partial x} & \frac{\partial X}{\partial y} \\ \frac{\partial Y}{\partial x} & \frac{\partial Y}{\partial y}\end{matrix} ] .$$

At any given point of the model the radial direction is defined by unit normal \hat{n}(u). The direction perpendicular to the radial direction is referred to as the cross-radial direction (In the case of short-axis slices the cross-radial direction is the circumferential direction. In the case of long-axis slices the cross-radial direction is the longitudinal direction). Using the model equations from the section describing the 2D method it can be shown that the radial strain is:

$${E}_{r} = \frac{1}{2} [ {( \frac{\partial Γ}{\partial γ} )}^{2} - 1 ] with \frac{\partial Γ}{\partial γ} = \frac{c ( u ) + γ d ( u )}{C ( u ) + Γ D ( u )} . \qquad ( 30 )$$

The cross-radial strain can be shown to be

$${E}_{c} = \frac{1}{2} [ \frac{{\vert \frac{d M}{d u} + \frac{\partial Γ}{\partial u} \hat{N} + Γ \frac{d \hat{N}}{d u} \vert}^{2}}{{\vert \frac{d m}{d u} + γ \frac{d \hat{n}}{d u} \vert}^{2}} - 1 ] \qquad ( 31 )$$

3D Strain

In the 3D case, if the mapping from the reference to the current frame in Cartesian coordinates is given by mapping functions X(x,y,z), Y(x,y,z) and Z(x,y,z), then the deformation gradient tensor is

$$F = [ \begin{matrix}\frac{\partial X}{\partial x} & \frac{\partial X}{\partial y} & \frac{\partial X}{\partial z} \\ \frac{\partial Y}{\partial x} & \frac{\partial Y}{\partial y} & \frac{\partial Y}{\partial z} \\ \frac{\partial Z}{\partial x} & \frac{\partial Z}{\partial y} & \frac{\partial Z}{\partial z}\end{matrix} ] \qquad ( 32 )$$

The mapping functions, written in vector form, can be expressed in terms of the displacement function u(r) given by (7)

$$[ \begin{matrix}X ( r ) \\ Y ( r ) \\ Z ( r )\end{matrix} ] = r + u ( r ) ,$$

where r=(x,y,z). Once Eq. (32) is evaluated, it can be used to evaluate Eq. (22) and then the radial, circumferential and longitudinal strains are computed by Eq. (29) using the respective directions.

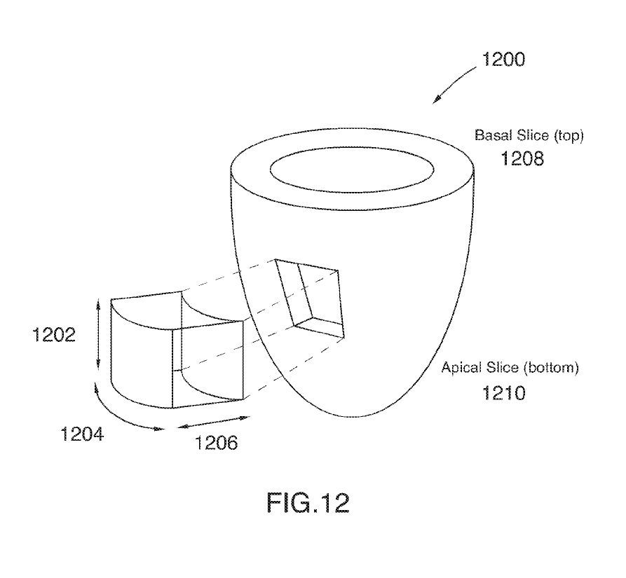

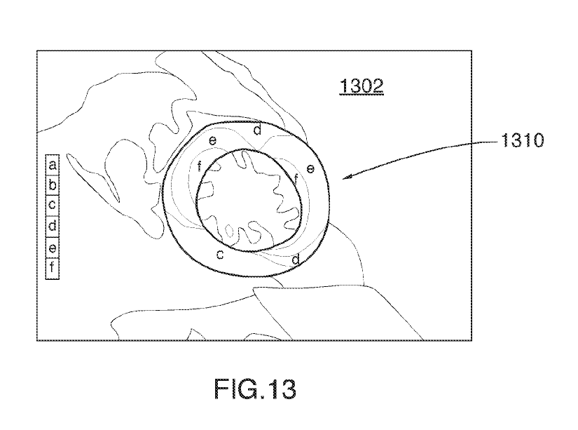

FIG. 12 shows a simplified model 1200 of the left ventricle illustrating various 3D strain directions. In particular, FIG. 12 illustrates the longitudinal 1202, circumferential 1204, and radial 1206 strain directions. FIG. 12 also illustrates the location of the Basal Slice 1208 (top portion of the model) as well as the apical slice 1210 (bottom portion of the model).

Post Processing of the Methods and Obtaining Statistical Results

After the 2D/3D models are completed the strains and displacements can be computed at each point in the myocardium.

Statistical results can be obtained:

1. Averaged radial/circumferential/long strain or displacements over a myocardium/subendo/subepi/a given ROI (diagrams)

2. Peak strain, peak displacements (in polarmaps)

3. Torsion

4. Volumes (endo/epi/myocardium) can be computed for the interpolated endo/epi surfaces. Myocardium volume is the difference of those two.

Reference is now made to FIGS. 13, 14, 15, and 16 which illustrate different types of displays that can be generated based on the methods and systems disclosed herein. FIG. 13 shows a SAX slice 1302 with a circumferential map 1310 obtained from part way through the cardiac cycle and is color-coded in relation to strain values. While any phase can serve as a reference frame or configuration, conventionally the configuration of the LV wall at the ED is chosen as the reference frame. The terms frame and phase are used interchangeably throughout the present disclosure.

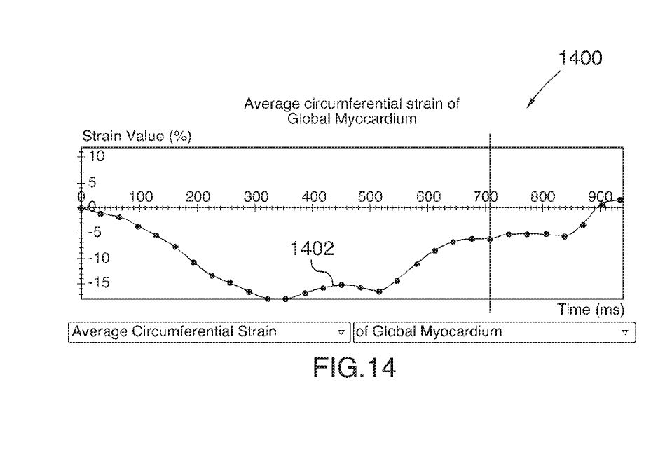

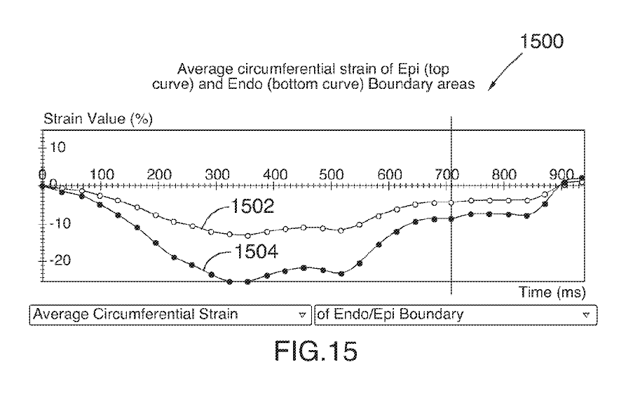

FIGS. 14 and 15 illustrate graphs 1400 and 1500, respectively, showing various strain curves. FIG. 14 illustrates curve 1402, which represents the average circumferential strain of the global myocardium. FIG. 15 illustrates two curves. Curve 1502 represents the average circumferential strain of the epicardial boundary and curve 1504 represents the average circumferential strain of the endocardial boundary.

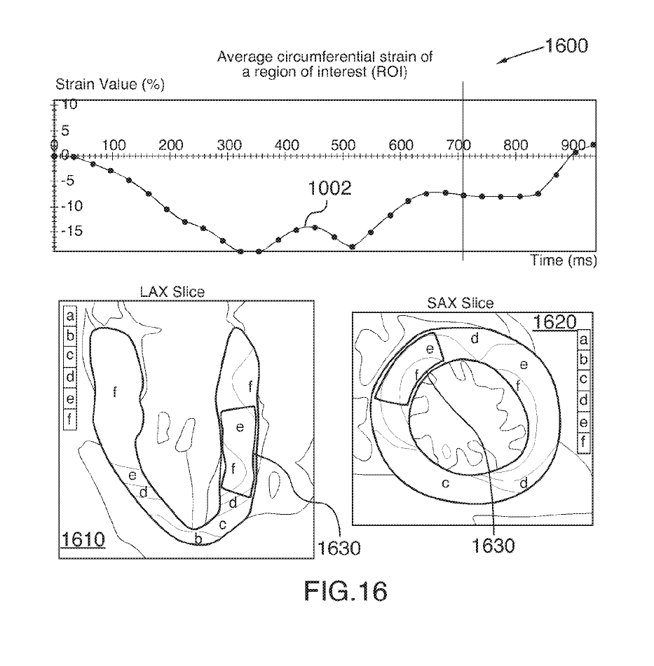

FIG. 16 illustrates a graph 1600, a LAX slice 1610, and a SAX slice 1620. Graph 1600 illustrates curve 1602 which represents the average circumferential strain of a region of interest (ROI) 1630. ROI 1630 is a three-dimensional section of the myocardium and its location is illustrated in each of LAX slice 1610 and a SAX slice 1620.

Reference is now made to FIG. 17, which illustrates screen 1700 which may be displayed on for example display 110 of system 10. Screen 1700 provides another example of the types of information that can be displayed once the model has been determined.

Screen capture 1700 includes a 3D view 1702 of a portion of the heart, which in the illustrated example is the left ventricle of the heart. View 1702 provides a 3D view of the strain over the portion of the myocardium of the heart. Screen capture 1700 also includes charts 1704 which provide information regarding the peak radial, circumferential, and longitudinal strain. Screen capture 1700 also includes a cross sectional (SAX slice) view 1706 of the myocardium that illustrates the strain over the cross section. Screen capture 1700 also includes graphs 1708 which illustrate various strain curves. The information used in the various portions of screen 1700 is generated based on the model and subsequent strain calculations.

As mentioned above the models and method disclosed herein may be used to identify areas of mechanical delay, myocardial insufficiency and dyssynchrony, as well as the spatial location of the tissue type of interest (for example, fibrosis, including diffuse fibrosis, related pathology as well as scar tissue, edema, or other tissues or tissue characteristics). The tissue characteristics can be either acute or chronic. This information can be used in the preparation for and execution of medical interventions and surgical procedures. For example, but not limited to, the above described displays can be useful in planning electrophysiological procedures.

Other Uses of the 2D/3D Methods

The above methods can be used to as contour detectors for endo/epi

This can be achieved directly from the endo/epi points tracked by the 2D method.

In addition, the endo/epi points from all of the slices that are tracked by the 2D method can be interpolated to define or generate surfaces. These surfaces can then be intersected with the image planes. These intersections can serve as detected endo/epi contours.

In addition, the 3D tracked surfaces intersect with image planes and define contours.

The above methods can also be used as contour detectors for RV Insertion point and LaxLvExtent or any other set of points on the myocardium.

Adjustment of the 2D Results Based on 3D Model

Once the 3D model is complete, it can be used to adjust the 2D results. To accomplish this, the in-slice endo/epi/myo points are converted from the reference frame to 3D coordinates and the directions used for computing the 2D strains (normal/tangential to the midcurve in reference frame) to 3D vector directions. The points are tracked by 3D method and the strains are evaluated. The diagrams/polarmaps/overlays that are used for diagnosis can then be updated with the adjusted results.

### APPENDIX

### Smooth Surface Model

We model the surfaces with pseudo thin plate splines defined on the sphere (see e.g. Wahba G., Spline interpolation and smoothing on the sphere, 1981, which is incorporated by reference in its entirety and is hereinafter referred to as Wahba). The closed form expression for the smoothest interpolator of arbitrary located data points on the sphere does not (Wahba). An approximation of the smoothest interpolator is referred to as pseudo thin plate splines. Wahba proposed a class of pseudo thin plate splines on the sphere and provided the corresponding closed form expression, which has the following form:

$$f ( \hat{u} ) = {α}_{0} + {\sum }_{n = 1}^{N} {α}_{n} ψ ( \hat{u} \cdot {\hat{u}}_{n} ) \qquad ( 1 )$$

In Eq. (27) û represents a unit vector (i.e. a direction in which the function needs to be evaluated), ûₙ represent a set of N unit vectors, α₀, . . . , α_{N} are model coefficients, and function ψ:[−1,1] [symbol] R defines the type of the pseudo thin plate splines. We use ψ for the case of m=2 in Wahba i.e.

$$ψ ( x ) = \frac{1}{2 π} [ \frac{1}{2} q ( x ) - \frac{1}{6} ] \\ where \\ q ( x ) = \{ \begin{matrix}[ \begin{matrix}( 12 {W}^{2} - 4 W ) ln ( 1 + \frac{1}{\sqrt{W}} ) - \\ 12 W \sqrt{W} + 6 W + 1\end{matrix} ] / 2 & - 1 \leq x < 1 \\ \frac{1}{2} & x = 1\end{matrix} \\ and W = \frac{1 - x}{2} \qquad ( 2 )$$

While Wahba proposed to use the model given by Eq. (27) as an interpolator, here it used as an approximator. To use the model as an approximator, the sphere is uniformly sampled with N=1000 unit vectors ûₙ. Let \vec{v}ₘ=vₘ\hat{v}ₘ m=1, . . . , M, represent the boundary points to be approximated with the surface model. The goal is to determine the coefficients of the model given by Eq. (27) that result in a smooth surface that approximates the boundary points as closely as possible. The above requirements result in the following optimization problem: find coefficients α₀, . . . , α_{N} that minimize

$$S = \frac{1}{M} {\sum }_{m = 1}^{M} {[ f ( {\hat{v}}_{m} ) - {v}_{m} ]}^{2} + λ \frac{1}{N} {\sum }_{n = 1}^{N} {α}_{n}^{2} \qquad ( 3 )$$

The first term corresponds to matching the boundary points and the second term controls the smoothness of the surface. Parameter λ controls the importance of the smoothness (second) term relative to the point matching (first) term. Note that both terms are normalized (divided by the number of terms in the respective summations), which means that λ does not need to be adjusted from case to case just because they have different numbers of boundary points. In order to minimize S, derivatives are taken with respect to the model coefficients and they are set equal to zero, i.e.

$$\frac{\partial S}{\partial {α}_{i}} = 0 i = 0 , … , N \qquad ( 4 )$$

This leads to:

$${α}_{0} + {\sum }_{n = 1}^{N} {α}_{n} [ \frac{1}{M} {\sum }_{m = 1}^{M} ψ ( {\hat{v}}_{m} \cdot {\hat{u}}_{n} ) ] = \frac{1}{M} {\sum }_{m = 1}^{M} {v}_{m} \\ {α}_{0} [ \frac{1}{M} {\sum }_{m = 1}^{M} ψ ( {\hat{v}}_{m} \cdot {\hat{u}}_{i} ) ] + {\sum }_{n = 1}^{N} {α}_{n} [ \frac{1}{M} {\sum }_{m = 1}^{M} ψ ( {\hat{v}}_{m} \cdot {\hat{u}}_{n} ) ψ ( {\hat{v}}_{m} \cdot {\hat{u}}_{i} ) ] + \frac{λ}{N} {α}_{i} = \frac{1}{M} {\sum }_{m = 1}^{M} {v}_{m} ψ ( {\hat{v}}_{m} \cdot {\hat{u}}_{i} ) for i = 1 , … , N \qquad ( 5 )$$

The N+1 equations (31) form a linear system for the N+1 unknowns α₀, . . . , α_{N}.

In the above system the surface point in direction û is ƒ(û)û.

In the preceding description, for purposes of explanation, numerous details are set forth in order to provide a thorough understanding of the embodiments. However, it will be apparent to one skilled in the art that these specific details are not required. Features described with respect one example embodiment may be implemented in another example embodiment, as appropriate. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto.

1. A method of determining characteristics of a myocardium using a model of the myocardium and a cine data set, the method comprising:
defining a 2D model of the myocardium;
determining the 2D model by fitting the 2D model to the cine data set;
defining a 3D model of the myocardium;
determining the 3D model based on data from the determined 2D model; and
performing post processing on the 3D model to determine myocardium characteristics.

2. The method of claim 1, wherein determining the myocardium characteristics comprises identifying tissue characteristics.

3. The method of claim 2, wherein identifying tissue characteristics comprises identifying fibrosis.

4. The method of claim 2, wherein identifying tissue characteristics comprises identifying edema.

5. The method of claim 2, wherein the tissue characteristic comprises an acute or chronic state.

6. The method of claim 1, wherein the myocardium characteristics comprise myocardial dynamics.

7. The method of claim 1, wherein the myocardium characteristics comprise myocardial strain.

8. The method of claim 1, wherein the method further comprises rendering a display of a 3D model of the myocardium, the 3D model including a display of strain information on the 3D model.

9. The method of claim 8, wherein the display of strain information comprises a graphical display of magnitudes of strain at various locations on the 3D model.

10. The method of claim 1, wherein the data from the determined 2D model comprises tracked endocardial and epicardial boundaries.

11. The method of claim 1, wherein the data from the determined 2D model comprises in-slice 2D displacements.

12. The method of claim 1, wherein determining the 2D model comprises:
identifying epicardial and endocardial contours in a reference frame of a cine data set;
identifying sample points in the reference frame;
tracking the sample points through each frame of the cine data set; and
determining the 2D model based on the tracked nodes.

13. The method of claim 12, wherein identifying sample points comprises:
identifying epi-points, endo-points, and midpoints based on the identified contours of the reference frame; and
wherein tracking the sample points through each frame comprises:
for each frame in the cine data set:
identifying points in the frame corresponding to epi-points and endo-points of a previous frame;
transferring midpoints to the frame from the previous frame; and
spatially translating the transferred midpoints to improve a match between the identified points in the frame with the corresponding epi-points and endo-points of the previous frame.

14. The method of claim 1, wherein determining the 3D model comprises:
defining surfaces to represent the myocardial wall reference frame;
setting up node coefficients of a surface model by selecting a set of control nodes from the defined surfaces;
selecting a set of myocardium points in a reference frame of the cine data set to serve as 2D sample points;
obtaining, for each of the 2D sample points, a set of 2D displacements from the determined 2D model;
defining a distance function to measure a total distance between the set of 2D displacements and a set of 2D projection of 3D displacements given by the 3D model;
defining a cost function based on the distance function and a smoothness of a displacement field of the 3D model; and
determining coefficients of the 3D model by minimizing the cost function.

15. The method of claim 1, wherein determining the 3D model comprises:
defining surfaces to represent the myocardial wall at the reference frame;
setting up node coefficients of a surface model by selecting a set of control nodes from the defined surfaces;
defining standard surfaces using endocardial and epicardial contours from the cine data set;
for each frame in the cine data set, generating an estimate of tracked nodes by projecting onto the
frame nodes of a previous frame and using the projections as the estimate of the tracked nodes;
defining a cost function to measure a distance between the tracked nodes and radial projections of the tracked nodes on the standard surfaces; and
determining coefficients of the 3D model by minimizing the cost function.

16. A method of determining characteristics of a myocardium using a 2D model of the myocardium and a cine data set, the method comprising:
identifying epicardial and endocardial contours in a reference frame of the cine data set;
identifying sample points in the reference frame;
tracking the sample points through each frame of the cine data set; and
performing post processing on the 2D model to determine myocardium characteristics.

17. The method of claim 16, wherein the myocardium characteristics comprise myocardial strain.

18. The method of claim 16, wherein the method further comprises rendering a display of a 2D model of the myocardium, the 2D model including a display of strain information on the 2D model.

19. The method of claim 18, wherein the display of strain information comprises a graphical display of magnitudes of strain on the 2D model.

20. The method of claim 16, wherein identifying sample points comprises:
identifying epi-points, endo-points, and midpoints based on the identified contours of the reference frame; and
wherein tracking the sample points through each frame comprises:
for each frame in the cine data set
identifying points in the frame corresponding to epi-points and endo-points of a previous frame;
transferring midpoints to the frame from the previous frame; and
spatially translating the transferred midpoints to improve a match between the identified points in the frame with the corresponding epi-points and endo-points of the previous frame.

21. A system for determining characteristics of a myocardium using a model of the myocardium and a cine data set, the system comprising:
a display,
an input device; and
a processor configured and adapted to:
define a 2D model of the myocardium;
determine the 2D model by fitting the 2D model to the cine data set;
define a 3D model of the myocardium;
determine the 3D model based on data from the determined 2D model; and
perform post processing on the 3D model to determine myocardium characteristics.

22. The system of claim 21, wherein determining the myocardium characteristics comprises identifying tissue characteristics.

23. The system of claim 22, wherein identifying tissue characteristics comprises identifying fibrosis.

24. The system of claim 22, wherein identifying tissue characteristics comprises identifying edema.

25. The system of 22, wherein the tissue characteristic comprises an acute or chronic state.

26. The system of claim 21, wherein the myocardium characteristics comprise myocardial dynamics.

27. The system of claim 21, wherein the myocardium characteristics comprise myocardial strain.

28. The system of claim 21, wherein the processor is further configured to render on the display a 3D model of the myocardium, the 3D model including a rendering of strain information on the 3D model.

29. The system of claim 28, wherein the display of strain information comprises a graphical rendering of magnitudes of strain at various locations on the 3D model.

30. The system of claim 21, wherein the data from the determined 2D model comprises tracked endocardial and epicardial boundaries.

31. The system of claim 21, wherein the data from the determined 2D model comprises in-slice 2D displacements.

32. The system of claim 21, wherein determining the 2D model comprises:
identifying epicardial and endocardial contours in a reference frame of a cine data set;
identifying sample points in the reference frame;
tracking the sample points through each frame of the cine data set; and
determining the 2D model based on the tracked nodes.

33. The system of claim 32, wherein identifying sample points comprises:
identifying epi-points, endo-points, and midpoints based on the identified contours of the reference frame; and
wherein tracking the sample points through each frame comprises:
for each frame in the cine data set:
identifying points in the frame corresponding to epi-points and endo-points of a previous frame;
transferring midpoints to the frame from the previous frame; and
spatially translating the transferred midpoints to improve a match between the identified points in the frame with the corresponding epi-points and endo-points of the previous frame.

34. The system of claim 21, wherein determining the 3D model comprises:
defining surfaces to represent the myocardial wall reference frame;
setting up node coefficients of a surface model by selecting a set of control nodes from the defined surfaces;
selecting a set of myocardium points in a reference frame of the cine data set to serve as 2D sample points;
obtaining, for each of the 2D sample points, a set of 2D displacements from the determined 2D model;
defining a distance function to measure a total distance between the set of 2D displacements and a set of 2D projection of 3D displacements given by the 3D model;
defining a cost function based on the distance function and a smoothness of a displacement field of the 3D model; and
determining coefficients of the 3D model by minimizing the cost function.

35. The system of claim 21, wherein determining the 3D model comprises:
defining surfaces to represent the myocardial wall at the reference frame;
setting up node coefficients of a surface model by selecting a set of control nodes from the defined surfaces;
defining standard surfaces using endocardial and epicardial contours from the cine data set;
for each frame in the cine data set, generating an estimate of tracked nodes by projecting onto the frame nodes of a previous frame and using the projections as the estimate of the tracked nodes;
defining a cost function to measure a distance between the tracked nodes and radial projections of the tracked nodes on the standard surfaces; and
determining coefficients of the 3D model by minimizing the cost function.

36. A system for determining characteristics of a myocardium using a 2D model of the myocardium and a cine data set, the system comprising:
a display,
an input device; and
a processor configured and adapted to:
identify epicardial and endocardial contours in a reference frame of the cine data set;
identify sample points in the reference frame;
track the sample points through each frame of the cine data set; and
perform post processing on the 2D model to determine myocardium characteristics.

37. The system of claim 36, wherein the myocardium characteristics comprise myocardial strain.

38. The system of claim 36, wherein the method further comprises rendering a display of a 2D model of the myocardium, the 2D model including a display of strain information on the 2D model.

39. The system of claim 38, wherein the display of strain information comprises a graphical display of magnitudes of strain on the 2D model.

40. The system of claim 36, wherein identifying sample points comprises:
identifying epi-points, endo-points, and midpoints based on the identified contours of the reference frame; and
wherein tracking the sample points through each frame comprises:
for each frame in the cine data set
identifying points in the frame corresponding to epi-points and endo-points of a previous frame;
transferring midpoints to the frame from the previous frame; and
spatially translating the transferred midpoints to improve a match between the identified points in the frame with the corresponding epi-points and endo-points of the previous frame.

41-43. (canceled)
